(19) United States
(12) Patent Application Publication
(10) Pub. No.: US 2025/0241898 A1
(43) Pub. Date: Jul. 31, 2025

(54) Topical Benzimidazole Formulations and Methods for Use in Treating Inflammatory Dermatoses

(71) Applicant: JJR&D, LLC, Celina, TN (US)

(72) Inventors: Joaquin J. Jimenez, Miami, FL (US); Naiem T. Issa, Selwood Pl, VA (US); John P. McCook, Austin, TX (US)

(21) Appl. No.: 19/180,752

(22) Filed: Apr. 16, 2025

Related U.S. Application Data
(63) Continuation-in-part of application No. 18/212,065, filed on Jun. 20, 2023.
(60) Provisional application No. 63/635,923, filed on Apr. 18, 2024.
(60) Provisional application No. 63/650,567, filed on May 22, 2024.
(60) Provisional application No. 63/353,907, filed on Jun. 21, 2022.

(51) Int. Cl. A61K 31/4184; A61K 9/06; A61K 47/10; A61K 47/14; A61K 47/22; A61K 47/38; A61K 47/40; A61P 17/00

(52) U.S. Cl. CPC A61K 31/4184; A61K 9/06; A61K 47/10; A61K 47/14; A61K 47/22; A61K 47/38; A61K 47/40; A61P 17/00

(57) ABSTRACT

Compositions and methods for treating and preventing inflammatory and autoimmune skin conditions, particularly rosacea, using one or more topically applied benzimidazole compounds in a pharmaceutically acceptable carrier for use on skin. A preferred benzimidazole compound comprises mebendazole. A treatment composition preferably comprises 0.05-0.075 weight percent mebendazole, and may comprise up to 20.0% mebendazole, in an aqueous carrier or vehicle comprising a cream, gel, lotion, liquid, emulsion, aerosol spray, non-aerosol spray,, suspension, or ointment and is applied at least once daily over a treatment period of at least two weeks to result in a reduction of cutaneous cytotoxic CD+8 T-cells, papules, pustules, swelling, appearance of redness or inflammation, and/or reduction in itchiness, and hot or burning sensation in the affected area compared to pre-treatment levels.

### CROSS REFERENCE TO RELATED APPLICATION

This application claims the benefit of U.S. Provisional Application Ser. No. 63/635,923 filed on Apr. 18, 2024 and U.S. Provisional Application Ser. No. 63/650,567 filed on May 22, 2024, which are incorporated herein by reference. This application is also a continuation in part of U.S. Ser. No. 18/212,065 filed on Jun. 20, 2023, which claims the benefit of U.S. Provisional Application Ser. No. 63/353,907 filed on Jun. 21, 2023, which are incorporated herein by reference.

### BACKGROUND OF THE DISCLOSURE

### 1. Field of the Invention

The present invention relates to topical formulations of benzimidazoles, and specifically of mebendazole, and their use in treating and preventing inflammatory and other skin diseases and conditions, such as rosacea.

### 2. Description of Related Technology

The use of benzimidazoles, including mebendazole, for treatment of various health related conditions in humans and non-human animals is known in the art. Mebendazole is an approved anthelminthic drug with favorable toxicity profile. It is generally given orally for intestinal helminthiasis. Dosing regimens have ranged from short-term low-dose treatments to long-term high-dose treatments over several months. For example, U.S. Pat. Nos. 9,877,950 and 5,169,846 disclose the use of mebendazole as an anthelminthic for treatment of parasites and worms in animals.

Interestingly, mebendazole has also been shown to have significant antiproliferative activity across various cancer models. These include glioblastoma, breast, lung, ovarian, colon, osteosarcoma, melanoma cell lines among others. Clinical trials are currently assessing the utility of mebendazole for high-grade glioma, medulloblastoma and metastatic gastrointestinal cancer. For example, WO2002058697 discloses the use of mebendazole for the treatment of cancers, including through topical application. Similarly, US20210369679 discloses the use of mebendazole with a non-steroidal anti-inflammatory (NSAID) for treating cancer, including skin cancer.

The anti-helminthic effect of mebendazole is attributed to its inhibition of tubulin, which disrupts the organism's cytoskeletal network leading to parasite death. Mebendazole also inhibits mammalian tubulin but with lesser binding affinity. It is believed that inhibition of tubulin is also one mechanism by which mebendazole exerts a cytotoxic effect on tumor cells. In addition to its ability to bind and inhibit tubulin polymerization, mebendazole also binds and inhibits numerous cytoplasmic kinases that are critical to cancer initiation, progression and metastasis. Examples of these kinases include ABL1, BRAF, DYRK1B, JAK3, and PDGFR.

The immunomodulatory effects of mebendazole have been cursorily studied in in vitro models. For example, mebendazole has been found to cause an M2 to M1 phenotypic switch in monocyte/macrophage models. This could potentially explain the benefit of mebendazole in solid malignancies beyond its direct anti-proliferative effect on tumor cells as the M1 macrophages induce direct and indirect anti-tumor effects. Furthermore, mebendazole has been shown to uniquely upregulate ERK signaling in THP-1 monocytoid cells and human CD4+ T-cells isolated from patients with systemic lupus erythematosus with known defective ERK signaling, an effect that is unique to mebendazole and not the other benzimidazoles. It is therefore postulated that mebendazole may be of use in autoimmune diseases characterized by defective ERK signaling. Mebendazole treatment of scleroderma is disclosed, for example, in WO2019112031. It has not, however, been known to use benzimidazoles for the topical treatment of other inflammatory or autoimmune diseases and conditions of the skin, such as rosacea.

Rosacea is one of the most common inflammatory skin disorders in humans. Rosacea is a chronic inflammatory skin disorder characterized by facial flushing, telangiectasias, irritation, pain, papules, pustules and phymatous/granulomatous changes. There are four major subtypes: erythematotelangiectactic rosacea (ETR), papulopustular rosacea (PPR), phymatous rosacea (PR), and ocular rosacea (OR). While initially thought to be vascular-driven disorder, rosacea is now appreciated to have a major immunologic component driving the disease process. This immunologic component is driven by numerous processes: pro-inflammatory neuropeptide release by cutaneous nerve endings, changes in the inflammatory milieu by the local microbiome, components of resident innate immunity altering the cytokine milieu, among others. With respect to adaptive immunity, presence and proliferation of CD4+ and CD8+ T-cells have been shown to be increased in ETR and PPR compared to healthy skin.

It is postulated that reduction of cutaneous inflammation leads to improvement of rosacea. This is indeed demonstrated by the use of topical and systemic anti-microbials, corticosteroids and other non-steroidal immunomodulators. Examples of such treatments include but are not limited to ivermectin, metronidazole, tetracyclines, prednisone, among others. As CD8+ T-cells are up-regulated in rosacea, it is also postulated that reducing/preventing the activity, proliferation or viability of CD8+ T-cells is a viable treatment strategy.

While the immunomodulatory effects of mebendazole have been investigated in monocytes/macrophages and CD4+ T-cells, its effect on CD8+ T-cells has not previously been investigated. There is a need in the art for an improved immunomodulatory treatment for inflammatory or autoimmune diseases and conditions of the skin in humans, and particularly treatment of rosacea. According to some embodiments of the disclosure, use of mebendazole results in such improvements, particularly in immunomodulatory effects on CD8+ T-cells.

There is also a need in the art for a mebendazole treatment composition that may be used for treating such skin conditions that has improved solubility and skin penetration ability. Mebendazole is poorly soluble in water with an aqueous solubility of approximately 0.035 mg/mL for mebendazole Polymorph C at 25° C. In addition to negligible solubility in water, mebendazole has negligible solublility in most solvents other than DMSO (approximately 2% by weight). Mebendazole is also freely soluble in formic acid. However, DMSO and formic acid are not suitable as pharmaceutical vehicle ingredients for a skin treatment product, particularly a facial skin treatment composition (as rosacea primarily impacts the face), due to safety considerations. Thus, there is a need for a process and composition ingredients to increase the solubility of mebendazole in a pharmaceutical vehicle that would be appropriate for use on human and animal skin, particularly human facial skin.

### SUMMARY OF THE DISCLOSURE

According to some embodiments, a treatment composition comprises one or more benzimidazoles in a pharmaceutically acceptable vehicle for topical application and may be used for the treatment of an inflammatory or autoimmune disease and/or condition of the skin. According to some embodiments, the disease or condition treated with a composition according to various embodiments herein is one affecting only animals (non-humans), only humans, or both animals and humans. Most preferably, compositions according to embodiments herein are used for the treatment of rosacea in humans.

In some embodiments, a treatment composition is in a form of a full solution, wherein all of the one or more benzimidazoles are fully dissolved. In other embodiments, a treatment composition is in a form of a full suspension, wherein all of the one or more benzimidazoles remains in an undissolved, suspended form. In still other embodiments, a treatment composition is partially a solution and a partially a suspension, wherein at least some of the one or more benzimidazoles are dissolved and at least some of the one or more benzimidazoles are undissolved and suspended in the composition.

According to some embodiments, a treatment composition comprises mebendazole or a salt of mebendazole or methyl 5-benzoylbenzimidazole-2-carbamate (each referred to herein generally as MBZ). In other embodiments, the treatment composition comprises one or more of albenzadole, thiabenzadole, fenbenzadole, MBZ, or salts of the foregoing. Except as referenced in the claims, any other benzimidazoles may be substituted for or used in addition to mebendazole or MBZ referenced herein in connection with the description of various embodiments. Mebenzadole exists in several polymorph crystalline states that include Polymorph A, B, and C. Polymorph C is the preferred form for some embodiments of compositions of herein, but the others may also be used or may be excluded. Salts of mebenzadole may include mebendazolium lactate, mebendazolium glycolate, and mebendazolium mesylate.

According to some embodiments, an MBZ treatment composition comprises around 0.01-1.0% mebendazole by weight, more preferably around 0.05-0.25% by weight, and most preferably around 0.01-0.075% by weight, in an MBZ gel composition, an MBZ cream base vehicle, or in another pharmaceutically acceptable vehicle for topical use with human and/or animal skin, with or without other active ingredients. In some embodiments, an MBZ treatment composition is in a form of a full solution, wherein all of the MBZ is fully dissolved. In other embodiments, an MBZ treatment composition is in a form of a full suspension, wherein all of the MBZ remains in an undissolved, suspended form. In still other embodiments, an MBZ treatment composition is partially a solution and partially a suspension, wherein at least some of the MBZ is dissolved and at least some of the MBZ is undissolved and suspended in the composition. Such amounts of MBZ are particularly preferred when the vehicle for the MBZ treatment composition is an aqueous or anhydrous solution, suspension or gel solution or suspension. According to another embodiment, the composition comprises (1) around 0.01-0.50% mebendazole by weight, more preferably around 0.05-0.25%, and most preferably around 0.01-0.075%; (2) around 10-60% by weight total of one or more solvents, penetrants, and/or emulsifying ingredients, more preferably around 25-60%, most preferably around 40-55%; and (3) water. One or more other active ingredients may also be included in these embodiments. In these embodiments where lower amounts of MBZ are used, the solvents and/or other ingredients preferably increase the solubility of MBZ to at least 5 times, more preferably at least 10 times or more of the solubility of MBZ in water, allowing less MBZ to be used to achieve increased skin penetration performance.

According to other embodiments, an MBZ treatment composition comprises a vehicle that does not increase the solubility of MBZ as much as other embodiments, thus requiring more MBZ to achieve to increase the skin penetration performance of such MBZ treatment compositions. One such vehicle is Vanicream® as described herein. In these embodiments, an MBZ treatment composition comprises 1.0-20.0% mebendazole by weight, more preferably around 1.0-10.0%, and most preferably around 1.0-5.0%. Such amounts of MBZ are particularly preferred when the vehicle for the MBZ treatment composition is an aqueous lotion, cream, emulsion or anhydrous suspension of mebendazole wherein the mebendozole is mostly in a non-soluble, crystalline form. In these embodiments where higher amounts of MBZ are used, the solvents and/or other ingredients preferably increase the solubility of MBZ to no more than 10 times, more preferably no more than 5 times or less of the solubility of MBZ in water.

According to certain embodiments, the mebendazole (or other benzimidazole) is physically nanosized in the composition. According to other embodiments, the mebendazole (or other benzimidazole) is also preferably suspended and/or non-solubilized mebendazole and is encapsulated in the composition. According to still other embodiments, the mebendazole (or other benzimidazole) is solubilized as much as possible without DMSO, decylmethylsulfoxide, dimethylacetamide, and pyrrolidone solvents, and/or surfactants. According to other embodiments, the mebendazole (or other benzimidazole) and at least one solvent are processed through a microfluidizer to form an MBZ (or other benzimidazole) concentrate composition and/or an MBZ (or other benzimidazole) treatment composition. According to another embodiment, the mebendazole (or other benzimidazole) and at least one solvent are not processed through a microfluidizer to form an MBZ (or other benzimidazole) concentrate composition and/or an MBZ (or other benzimidazole) treatment composition. These variations on the mebendazole (or other benzimidazole) may be used with any compositions comprising mebendazole (or other benzimidazole) according to embodiments of the compositions herein.

According to another embodiment, the vehicle in the composition comprises an aqueous lotion, cream, gel, solution, suspension, or ointment wherein the MBZ is present (1) fully dissolved form or (2) in both soluble (partially dissolved) and insoluble (partially undissolved) form, with the insoluble portion preferably suspended in the composition or (3) a fully suspended form. Embodiments for a vehicle may comprise one or more of the following, and in some preferred embodiments all of the following: (1) water (preferably purified or deionized), (2) petrolatum, (3) sorbitol, (4) cetearyl alcohol, (5) propylene glycol, (6) ceteareth-20 (a polyethylene glycol ether of cetearyl alcohol), (7) simethicone, (8) glyceryl stearate, (9) PEG-30 Stearate (a polyethylene glycol ester of stearic acid), (10) sorbic acid, and (11) BHT (butylated hydroxytoluene). Other embodiments for a vehicle comprise one or more of the following, preferably all of the following: (1) water (preferably purified or deionized), (2) glycerin, (3) polyacrylic acid (preferably carbomer), (4) disodium ethylenediaminetetraacetic acid (EDTA), (5) cetyl alcohol, (6) stearyl alcohol, (7) glyceryl stearate and/or PEG 100 stearate, (8) polysorbate 80, (9) triethanolamine, and (10) phenoxyethanol. Any ingredient listed as potentially included in an embodiment or in one preferred embodiment, may also be excluded in another embodiment or another preferred embodiment.

According to some embodiments, an MBZ concentrate composition comprises: (1) mebendazole (or other benzimidazole) and (2) one or more compounds or ingredients that act as solvents, skin penetrants, and/or emulsifying agents. According to another embodiment, there are at least two, more preferably at least three, ingredients in category (2). Preferably, at least one ingredient in category (2) acts as a solvent and an emulsifying agent. According to another embodiment, an MBZ concentrate composition comprises: (1) mebendazole (or other benzimidazole); (2) a sorbitol based solvent, (3) an ethylene and/or propylene glycol based solvent, and (4) a solubilizer and/or emulsifying agent. These MBZ concentrate compositions comprise around 1.0-10.0% by weight mebendazole, more preferably around 3.0-6.0%, most preferably around 3.0-4.0%. According to still other embodiments, the amount of mebendazole in an MBZ concentrate composition is reduced to around 0.05-1.0% by weight, more preferably 0.05-0.2%, and most preferably 0.01-0.075%.

A preferred ethylene glycol based solvent is diethylene glycol monoethyl ether (Transcutol P®), but others may also be used. A preferred propylene glycol based solvent is propylene glycol monolaurate (Lauryl Glycol®), but others may also be used. According to another embodiment, an MBZ concentrate composition comprises around 15.00-25.00% by weight total of the ethylene and/or propylene glycol based solvent, more preferably around 18.00-21.00%. In some embodiments, both an ethylene glycol based solvent and a propylene glycol based solvent are used together in an MBZ concentrate composition in weight ratios of around 80:20 to 70:30, more preferably around 75:25 to 65:35, and most preferably around 60:40.

A preferred sorbitol based solvent is dimethyl isosorbide (DMI). According to another embodiment, an MBZ concentrate composition comprises around 32.00-45.00% by weight of the sorbitol based solvent, more preferably around 36.00-40.00%.

A preferred solubilizer and/or emulsifying agent is polyoxyl 40 hydrogenated castor oil (Cremophor RH 40). According to another embodiment, an MBZ concentrate composition comprises around 32.00-45.00% by weight of the solubilizer and/or emulsifying agent, more preferably around 36.00-40.00%.

The solvents used in compositions according to some embodiments are (1) preferably capable of dissolving MBZ at 10× or more the solubility of MBZ in water (which is approximately 35 ug/mL) and (2) suitable for use, in amounts necessary to dissolve MBZ for topical application to human or animal skin. According to still other embodiments, other solvents/skin penetrants that may be used in addition to or as alternates to dimethyl isosorbide, diethylene glycol monoethyl ether, and Cremophor RH 40 include: caprylocaproyl macrogol-8 glycerides, propylene glycol monocaprylate, polyglyceryl-3 dioleate, diisopropyl adipate, diethyhexyl adipate, poly(glyceryl) adipate, salicylate esters, Cyrene (dihydrolevoglucosenone), Poloxamer 188, Poloxamer 407, Deep Eutectic Solvents or Natural Deep Eutectic Solvents which may contain one or more quarternary ammonium salt, quarternary phosphonium salt, urea, amide, carboxylic acid, alcohol, polyatomicalcohol, amine,glycerol, lactic acid, n-decanoic acid, choline chloride, tetrabutylammonium chloride, sucrose, sucrose ester, mono, di, or polysaccharide, and diethylene glycol monoethylether. According to some embodiments, a solvent used in compositions herein does not include an aprotic solvent, a polar aprotic solvent, a dipolar aprotic solvent, or a non-polar aprotic solvent. According to some embodiments, a polyol is not used as a solvent in compositions herein. In some embodiments, a dipolar aprotic solvent is not used in combination with a polyol in compositions herein. In still other embodiments, an aprotic solvent may be used in compositions according to some embodiments and the aprotic solvent may be polar, dipolar, or non-polar. In still other embodiments, a polyol may be used as a solvent in compositions according to some embodiment herein and may or may not be used in combination with a dipolar aprotic solvent. In still other embodiments, cyrene (dihydrolevoglucosenone) may be used in compositions in place of aprotic or non-aprotic solvents. In still other embodiments, cyrene may be excluded from compositions herein.

According to some embodiments, an MBZ concentrate composition according to some embodiments herein is made by: (1) mixing the one or more solvents and mebendazole to form a first mixture; (2) heating the first mixture to a temperature within a first temperature range for a first period of time; and (3) mixing or stirring during the heating step, such as by using a magnetic stirrer. According to some embodiments, the first period of time is around 5 to 20 minutes, more preferably around 5 to 15 minutes. Most preferably, the heating temperature and first period of time will not result in degradation of the MBZ of more than 10%, more preferably not more than 8% and most preferably not more than 5%. The first temperature range is most preferably between at least 60° C. but less than a temperature at which MBZ will experience degradation of 5% or more. More preferably, the first temperature range is 60-100° C., and most preferably 70-100° C. Preferably, steps 1-3 are carried out before mixing the MBZ or the first mixture with any water or aqueous based application vehicle.

According to some embodiments, an MBZ gel composition comprises (1) mebendazole (or other benzimidazole); (2) water, and (3) a gelling or thickening agent (or viscosity modifying agent). According to another embodiment, an MBZ gel composition comprises (1) an MBZ concentrate composition, (2) water, and (3) a gelling or thickening agent (or viscosity modifying agent). A preferred gelling/thickening/viscosity modifying agent is hydroxyethylcellulose, such as Natrosol® 250HX (pharma grade), but other agents may also be used. According to other embodiments, an MBZ gel composition further comprises (4) a cyclodextrin compound to aid in solubility and to reduce precipitation of the MBZ. A preferred cyclodextrin compound is hydroxypropyl beta cyclodextrin (Cavasol® W7 HP). Other cyclodextrin compounds comprising alpha-and gamma-cyclodextrins and alternate chemical modifications of the alpha, beta and gamma cyclodextrins in addition to hydroxyalkylation including, methylation, ethylation, phosphated and sulfonated, may also be used. According to still other embodiments, an MBZ gel composition further comprises (5) an ethylene glycol based solvent, such as diethylene glycol monoethyl ether (Transcutol P®).

According to other embodiments, an MBZ gel composition comprises (1) mebendazole (or other benzimidazole); (2) water, (3) a gelling or thickening agent (or viscosity modifying agent), and (4) an ethylene glycol-based solvent. According to another embodiment, an MBZ gel composition comprises (1) an MBZ concentrate composition, (2) water, (3) a gelling or thickening agent (or viscosity modifying agent), and (4) an ethylene glycol-based solvent. A preferred gelling/thickening/viscosity modifying agent is hydroxyethylcellulose, such as Natrosol® 250HX (pharma grade), but other agents may also be used. A preferred ethylene glycol-based solvent is diethylene glycol monoethyl ether (Transcutol P®), but others may also be used.

According to some embodiments, an MBZ gel composition comprises around 0.01-0.075% MBZ and around 15-45% ethylene glycol-based solvent. In some embodiments, an MBZ gel composition comprises around 0.04-0.07% MBZ and around 15-25% ethylene glycol-based solvent. In still other embodiments, an MBZ gel composition comprises around 0.04-0.06% MBZ and around 18-22% ethylene glycol based solvent. In some embodiments, an MBZ gel composition comprises around 0.5 to 1.5% of hydroxyehtylcellulose (a viscosity modifying agent). In some embodiments, an MBZ gel composition comprises around 15 to 25% of an ethylene glycol-based solvent.

In some embodiments, an MBZ gel composition comprises a ratio of ethylene glycol-based solvent, preferably Transcutol® P, to water of around 1:2 to 1:5, more preferably around 1:4. In some embodiments, an MBZ gel composition comprises a ratio of ethylene glycol based solvent, preferably Transcutol® P, to water of greater than 1:1.5. These percentages and ratios are by weight.

According to other embodiments, an MBZ treatment composition comprises an MBZ gel composition that may be mixed with a vehicle or applied to skin for treatment without mixing with additional vehicle ingredients. According to other embodiments, an MBZ treatment composition comprises an MBZ concentrate composition that is mixed with a vehicle prior to application to skin for treatment.

According to another embodiment, a method of treating an inflammatory or autoimmune disease and condition of the skin comprises topically applying a composition comprising one or more benzimidazoles in a pharmaceutically acceptable vehicle to an affected area of the skin at least once daily for a treatment period of at least two weeks, more preferably at least twelve weeks and continue to apply the composition to the affected facial area daily or every other day to prevent reoccurrence of a new rosacea outbreak. Most preferably, the composition used in methods according to embodiments herein is one according to a preferred embodiment. According to one method embodiment, a composition comprising mebendazole plus a vehicle is applied to the affected area of the skin at a dosage rate of around 100 to 1000 milligrams, more preferably 50 to 500 milligrams to each side of the face to the affected areas.

Use of treatment compositions and methods according to embodiments herein have been shown to be effective in reducing viability of T-cells and in treating a patient with rosacea. Treatment compositions according to embodiments herein have been shown to have greater skin permeation rates, including greater permeation in shorter period of time. The inventors also have found that use of mebendazole according to some embodiments reduced the viability of T-cells in an in vitro model. Use of preferred methods and composition ingredients for making an MBZ concentrate composition, an MBZ gel composition, and/or an MBZ treatment composition have also been shown to result in increased skin penetration and/or increased solubility of MBZ in a pharmaceutical vehicle appropriate for use on human and animal skin, particularly human facial skin. Accordingly, the methods and compositions of the embodiments herein, particularly preferred embodiments, provide advantages as a therapeutic agent for immunomodulation, particularly through a suppressive effect on CD8+ T-cells, for treatment of inflammatory and autoimmune diseases and conditions of the skin, particularly rosacea, with improved penetration and solubility of the MBZ.

### BRIEF DESCRIPTION OF THE DRAWINGS

The compositions and methods of the disclosure are further described and explained in relation to the following figures wherein:

FIG. 1 is a graph depicting the reduction in density of T-lymphocytes when exposed to various concentrations of a composition comprising mebendazole;

FIG. 2 is a graph depicting a reduction in the number of papules and pustules on the left (treated) cheek of a patient with rosacea when treated with a cream composition comprising 10% mebendazole by weight;

FIG. 3 contains photographs showing the left (treated) and right (untreated/control) cheeks of the patient with rosacea referenced in FIG. 2;

FIG. 4 is a graph depicting flux values at 0-6 hours compared to 6-24 hours for MBZ treatment compositions in Example 8;

FIG. 5 is a graph depicting flux values at 0-1 hours compared to 0-6 hours and compared to 6-24 hours for MBZ treatment compositions in different receptor fluids in Example 9;

FIGS. 6A-6B are tables showing data from Examples 6, 8, and 9; and

FIG. 7 is a more complete t-table for treatment compositions for Example 9.

### DETAILED DESCRIPTION

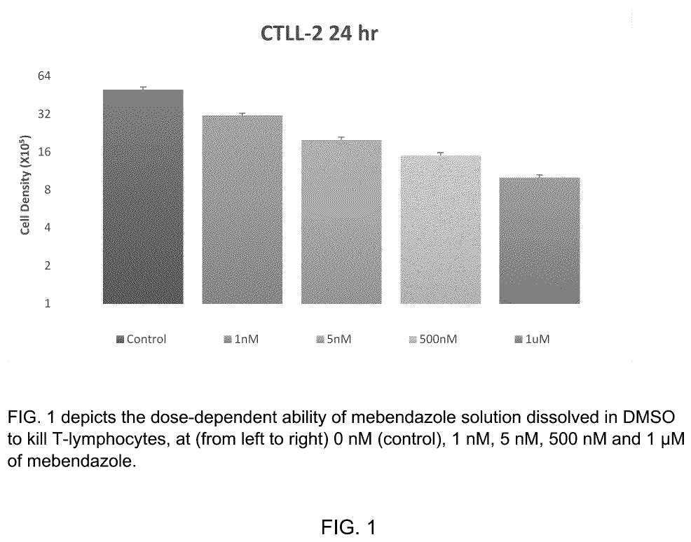

The mebendazole topical treatment compositions according to some embodiments of the disclosure are effective in the reduction of T-lymphocytes (see FIGS. 1-3), and can be used as effective immune modulators alone, as well as in combination with an additional therapeutic agent, such as oxymetazoline. Accordingly, disclosed herein are methods of treating or preventing a disease or disorder in a subject (preferably a human), comprising administering to the subject a therapeutically effective amount of a composition comprising one or more benzimidazoles in a vehicle or carrier for topical application. Most preferably, the benzimidazole comprises mebendazole, which is in a topical cream vehicle or a topical gel as described herein. Most preferably, the vehicle or carrier is a pharmaceutically acceptable carrier composition suitable to use on human and/or animal (non-human) skin. Preferably, the disease or disorder is an autoimmune disease or an inflammatory skin disorder. Most preferably, the disease or disorder is rosacea.

As used herein, the terms “treat,” “treating,” “treatment,” and the like refer to eliminating, reducing, or ameliorating a disease or condition, and/or symptoms associated therewith. Although not precluded, treating a disease or condition does not require that the disease, condition, or symptoms associated therewith be completely eliminated.

As used herein, the terms “treat,” “treating,” “treatment,” and the like may include “prophylactic treatment,” which refers to reducing the probability of redeveloping a disease or condition, or of a recurrence of a previously-controlled disease or condition, in a subject who does not have, but is at risk of or is susceptible to, redeveloping a disease or condition or a recurrence of the disease or condition. The term “treat” and synonyms contemplate administering a therapeutically effective amount of a composition of the disclosure to an individual in need of such treatment. Within the meaning of the disclosure, “treatment” also includes relapse prophylaxis or phase prophylaxis, as well as the treatment of acute or chronic signs, symptoms and/or malfunctions. The treatment can be orientated symptomatically, for example, to suppress symptoms. It can be affected over a short period, be oriented over a medium term, or can be a long-term treatment, for example within the context of a maintenance therapy. As used herein, the terms “prevent,” “preventing,” and “prevention,” are art-recognized, and when used in relation to a condition, such as rosacea, is well understood in the art, and includes administration of a composition which reduces the frequency of, or delays the onset of, symptoms of a medical condition in a subject relative to a subject which does not receive the composition. Thus, prevention of rosacea includes, for example, reducing inflammation, restoring or reducing the levels of T-cell lymphocytes near hair follicles in a treated population versus an untreated control population, e.g., by a statistically and/or clinically significant amount.

In some cases, the compositions and methods disclosed herein comprise those for treating or preventing rosacea. In some cases, the compositions and methods disclosed herein comprise those for treating rosacea. In some cases, the compositions and methods disclosed herein comprise those for preventing rosacea.

Preferred MBZ treatment compositions comprise around 0.01-1.0% mebendazole by weight, more preferably around 0.05-0.5%, and most preferably around 0.01-0.075%. Depending on the other ingredients used in MBZ treatment compositions, particularly the solvents or penetrants used, the amount of mebendazole may be increased to up to around 20.0% by weight, more preferably up to around 10.0%, and most preferably up to around 5.0% to allow for sufficient solubility and skin penetration of the mebendazole. The balance of preferred MBZ treatment composition comprise pharmaceutically acceptable carrier or vehicle ingredients for topical administration and optionally one or more other pharmaceutically active ingredients. A pharmaceutically active vehicle or carrier may comprise a lotion, cream, suspension, or ointment that is aqueous, or a clear or cloudy aqueous gel solution or gel suspension. One preferred vehicle is a moisturizing cream or lotion comprising an emulsion for facial treatment or prevention of erythematotelangiectactic rosacea, papulopustular rosacea, and phymatous rosacea, and a sterile ophthalmic solution, suspension, emulsion, or ointment for treatment or prevention of ocular rosacea, wherein the MBZ is in solution, in suspension or in both suspension and solution.

In some cases, the methods disclosed herein comprise administering to the subject one or more additional pharmaceutically active agents and/or such one or more additional pharmaceutically active agents may be included in a treatment composition according to embodiments herein. In some cases, the additional pharmaceutically active agent is an immunosuppressant, an anti-infective, calcineurin inhibitor, Janus kinase (JAK) inhibitor, retinoid or vasoconstricting agent. In some cases, the immunosuppressant is a corticosteroid. In some cases, the corticosteroid is hydrocortisone, triamcinolone, clobetasol, fluocinonide, etc. In some cases, the anti-infective agent is tetracycline, doxycycline, minocycline, erythromycin, metronidazole, ivermectin, etc. In some cases, the calcineurin inhibitor is cyclosporine, pimecrolimus or tacrolimus. In some cases, the JAK inhibitor is ruxolitinib, tofacitinib, baricitinib or oclacitinib. In some cases, the retinoid agent is azelaic acid, tretinoin, retinol. In some cases, the vasoconstricting agent is brimonidine, midodrine or oxymetazoline. In other embodiments, any of these ingredients may also be excluded from the composition or excluded from use in the methods of the disclosure. Chronic use of corticosteroids are excluded from the composition according to some embodiments as contraindicated.

Additionally, as the compositions and methods of some embodiments herein involve topical application to the skin or ocular membranes, other non-active excipients which may aid in or induce penetration of the active benzimidazole agent(s) into the facial skin or ocular tissue may be used. Substances termed “permeation enhancers,” are typically used in compositions designed to deliver drugs transdermally to increase the amount of the active that is delivered into the systemic circulation. Permeation enhancers constitute various classes of compounds including certain compounds such as dimethylsulfoxide (DMSO) or other organic sulfoxides, methylsulfonylmethane, pyrrolidones, ethanol, propylene glycol, dimethylacetamide (DMA), and others that are capable of disrupting the barrier function of the stratum corneum. In some embodiments, one or more of DMSO, other sulfoxides, DMA, pyrrolidones and other non-polar or polar aprotic solvents are used in compositions and methods herein, including topical formulations of benzimadazoles, including MBZ, to enhance skin penetration. In other embodiments, these ingredients are excluded from compositions and methods herein. Other substances have also been shown to increase the flux of certain active agents through the skin or mucous membranes. These include lipophilic compounds such as laurocapram (Azone); alpha-hydroxy and beta-hydroxy acids, fatty acids or alcohols such as oleic acid, oleyl alcohol, linoleic acid and the like; certain fatty acid esters such as isopropyl myristate, methyl nonanoate, methyl caprate and others. Mixtures of certain permeation enhancers with propylene glycol, butylene glycol, pentylene glycol, isopentyl glycol, hexylene glycol, diethylene glycol monoethyl ether; also known as ethoxydiglycol, acetamide MEA, and propylene glycol monolaurate are also known to improve the delivery of certain active ingredients. Certain surfactants may also increase penetration of active agents through solubilization and reduction in interfacial surface tension or encapsulation of the active agent and may include PEG-40 hydrogenated castor oil, Cremophor® or other surfactants combining glyceryl polyethylene glycol oxystearate, fatty acid glyceryl polyglyceryl esters, polyethylene glycol, and glyceryl ethoxylates. Encapsulation media for the benzimidazole drug substance or compositions according to some embodiments may also include soy or egg based lecithin or chemically modified lecithin, phosphatidylcholine or modified phosphatidylcholine, beta-cyclodextrins, PLGA [poly(lactic-co-glycolic acid], dextran, chitosan, d-alpha-tocopheryl polyethylene glycol succinate polymers and poloxamer (polyoxyethylene-polyoxypropylene) block polymers. Compositions according to some embodiments may comprise one or more of these ingredients or other permeation enhancers. In other embodiments, any of these ingredients may also be excluded from the composition or excluded from use in the methods of the disclosure. In still other embodiments, compositions herein may include one surfactant but do not include a second surfactant or cosurfactant.

Although numerous ingredients may be used with the compositions according to certain embodiments, according to other embodiments they are excluded. Compositions according to certain embodiments do not include NSAIDs, 2,6-di-tert-butyl-4-methylphenol, phenyl-pentanedione/or pyridine/or phenylbenzene/or benzoxazine compounds (such as 3-(4-methozyphenyl)-1,5-bis(2-methoxyphenyl)-1,5-pentanedione). Hyaluronic acid/salt may be included in some embodiments and may be excluded in other embodiments herein. One or more methyl sulfoxides, including without limitation DMSO and decylmethylsulfoxide, may be included in some embodiments and may be excluded in other embodiments herein. One or more pyrrolidones, including without limitation 2-pyrrolidone and N-mehtylpyrrolidone, may be included in some embodiments and may be excluded in other embodiments herein. One or more dipolar aprotic solvents, including without limitation DMSO and DMA, may be included in some embodiments and may be excluded in other embodiments herein. One or more disubstituted amides, including without limitation N,N-dimethylformamide, N,N-diethyltoluamide, or N,N-dimethylcaprylamide may be included in some embodiments and may be excluded in other embodiments herein. One or more linear alcohols, including without limitation ethanol, may be included in some embodiments and may be excluded in other embodiments herein. One or more branched alcohols, including without limitation isopropanol or 2-octyldodecanol type, may be included in some embodiments and may be excluded in other embodiments herein. One or more ketones, including without limitation acetone or methyl ethyl ketone, may be included in some embodiments and may be excluded in other embodiments herein. One or more alkyl ethers and/or alkyl esters, including without limitation ethyl lactate, may be included in some embodiments and may be excluded in other embodiments herein.

With respect to the amount of mebendazole incorporated in each topical formulation, the mebendazole content will typically be adjusted such that when the topical formulation is applied to a treatment area of a subject in need thereof, the amount of compound for reducing inflammation. (i.e., for treating rosacea) is present in an amount effective to achieve at least one of: (i) reduction of T-cell viability, (ii) reduction of T-cell proliferation, (iii) reduction of T-cell activation, (iv) reduction of T-cell activity.

Uses of the topical compositions disclosed herein in the preparation of a medicament for treating the diseases and disorders described herein are provided.

EXAMPLES. Described in the below Examples 1 and 2 are methods for evaluating mebendazole for efficacy in modulating T-lymphocytes in an in vitro platform translatable to human skin disease and its clinical application with a MBZ treatment composition according to a preferred embodiment of the disclosure. The results of these examples demonstrate that mebendazole is a promising immunomodulatory agent for treatment of rosacea and possibly other forms of inflammatory and neoplastic skin diseases driven by T-lymphocytes to ultimately improve the patient's quality of life. Described in the below Examples 3-9 are compositions and methods for making treatment compositions comprising MBZ according to some embodiments of the disclosure, along with solubility analysis of MBZ in select solvents and Skin PAMPA testing of select treatment compositions for evaluation of skin penetration. The following examples are provided for illustration and are not intended to limit the scope of the disclosure.

### Example 1—In Vitro Trial

An in vitro screening platform for immunomodulation was used to evaluate mebenzadole for efficacy. CTLL-2 (ATCC® TIB-214™) are cytotoxic T lymphocytes that were purchased and cultured according to ATCC protocol. Once confluent, cells were seeded in a 12-well plate at a concentration of 55,000 cells/mL and treated, in triplicates, with a solution of mebendazole dissolved in dimethyl sulfoxide (DMSO) at varying mebendazole concentrations of 1 nM, 5 nM, 500 nM and 1 μM for comparison to a control that had only DMSO applied (without any mebendazole). The maximum volume added to the 1 mL of each of the 12 wells was 10 μL for the highest test concentration of mebenzadole and the control. Although the concentrations of mebendazole are within preferred embodiments of treatment compositions herein, preferred treatment compositions for use do not include DMSO. MBZ has good solubility in DMSO (approximately 2% by weight), which is why it was selected as a solvent for this example; however, it is preferred to not use DMSO as a solvent in MBZ treatment compositions herein due to safety considerations with skin applications. As indicated in FIG. 1, mebendazole is shown to dose dependently kill T-lymphocytes. The results indicate that mebenzadole significantly reduces the T-lymphocytes, in cell culture, starting at a test concentration of 1 nM (0.295 micrograms).

In vitro data disclosed in above shows significant reduction of inflammatory CD+8 T cells starting at concentrations of 1 nM or 0.295 μg per mL in DMSO. This extremely low concentration for reduction of T-cells becomes important in formulating mebendazole (or methyl 5-benzoylbenzimidazole-2-carbamate) in a topical cream, lotion, gel, suspension, emulsion, patch or any topical pharmaceutical vehicle for application to human or mammalian skin and most preferably to human facial skin afflicted with rosacea.

### Example 2—Application of Mebendazole for the Treatment of Rosacea

Without wishing to be bound by theory in the immune model of rosacea, the inflammatory process induced by activated T-cells leads to the following changes that give rise to all sub-types of rosacea previously noted: angiogenesis, formation of papules and pustules, and granulomatous inflammation that leads to phymatous changes of the skin. The recovery process is complex. Again, without wishing to be bound by theory, the activated T-cells must be deactivated (e.g., with mebendazole) and then the skin achieves homeostatic normalcy that resolves the cutaneous erythema, telangiectasias, papules, pustules and phymatous changes.

A clinical study was conducted to determine the effect of 10% mebendazole cream according to one preferred MBZ treatment composition applied daily according to one preferred treatment method on the remission of rosacea after 12 weeks. In order to collect the preliminary information on the remission of rosacea, a split-face study was performed where the Patient applied the MBZ treatment composition (comprising 10% mebendazole by weight) to the left side of her face and the vehicle cream (as a control, without any mebendazole) to the right side of her face nightly for 12 weeks.

The Patient is a 52-year-old female with clinically diagnosed papulopustular rosacea (PPR) of the face over decades. She received numerous treatments including topical ivermectin, topical metronidazole, topical steroids, topical antibiotics and systemic antibiotics. After discontinuation of the aforementioned treatments, her rosacea would flare with erythema, telangiectasias, papules and pustules. The Patient underwent a 4-week washout period without use of topical or systemic active medications prior to initiating the active (10% mebendazole) and control (vehicle) creams. The amount of mebendazole in the cream used in Example 2 is equivalent to a molar concentration of 0.339M and approximately 0.25 to 0.5 mL of the cream was applied to the left cheek of the Patient once per day.

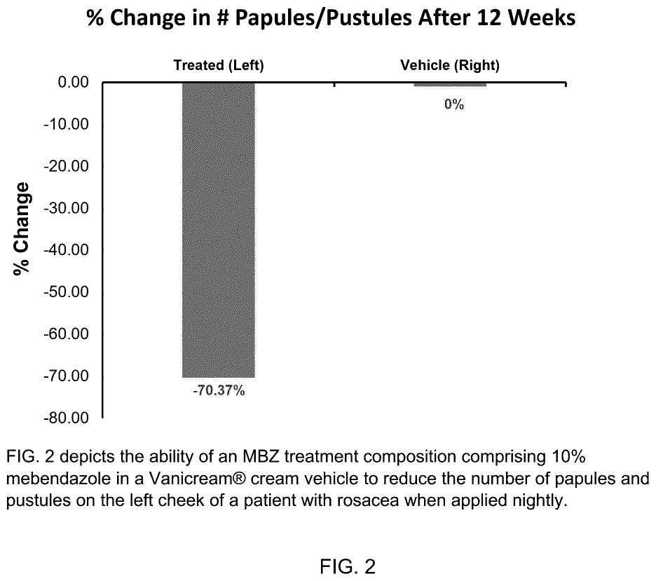

The results are summarized in FIG. 2. Reported categories are as follows: treated (10% mebendazole, left cheek) and control (vehicle, right cheek). In these results, active (10% mebendazole) showed a decrease in the total number of papules and pustules of the left face (70.37%) whereas the vehicle did not result in a decrease, indicating an improvement of the rosacea when treated according to a preferred embodiment of the disclosure.

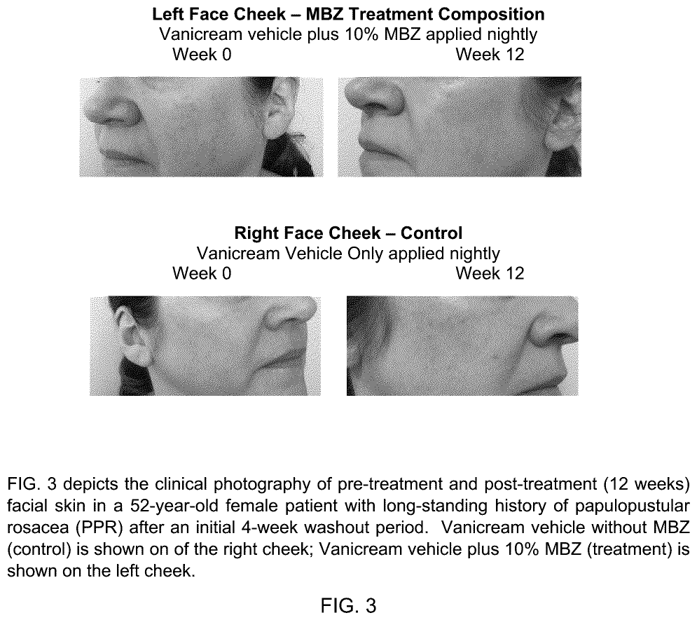

The results are also represented by photos in FIG. 3 depicting improvement of the rosacea of the left cheek with a 12-week nightly treatment with active (10% mebendazole) cream but no improvement of rosacea of the right cheek with 12-week nightly treatment with control (vehicle). Improvement is noted by reduction in papules and pustules as well as reduction in erythema and telangiectasias.

The MBZ treatment composition used in the treatment of the Patient was formulated by adding Mebenzadole Tablets, USP, 500 mg, crushed to a fine powder, mixed until uniform and in a sufficient amount to make a 10% by weight mebenzadole suspension in a commercially available Vanicream® Moisturizing Skin Cream vehicle. No attempt to increase the solubility of the MBZ in the commercial Vanicream® vehicle was made. The label ingredient listing (INCI; International Nomenclature Cosmetic Ingredient) for the Vanicream® Moisturizing Skin Cream, which is an oil-in-water emulsion cream base, is as follows: Purified water, petrolatum, sorbitol, cetearyl alcohol, propylene glycol, ceteareth-20 (a polyethylene glycol ether of cetearyl alcohol), simethicone, glyceryl stearate, PEG-30 Stearate (a polyethylene glycol ester of stearic acid), sorbic acid, BHT (butylated hydroxytoluene). MBZ is poorly soluble in water and it should be noted that the aqueous solubility of MBZ is approximately 0.035 mg/mL (Polymorph C at 25 degrees C.). In addition to negligible solubility in water, MBZ has negligible solublility in most solvents other than DMSO (approximately 2% by weight). MBZ is also freely soluble in formic acid. Neither DMSO nor formic acid is an appropriate pharmaceutical vehicle ingredient for a facial treatment product due to safety considerations. Thus, there is a need for a process to increase the solubility of MBZ in a pharmaceutical vehicle that would be appropriate for use on human and animal skin, particularly human facial skin.

### Example 3—Increased Penetration and Solubility Trials

Further laboratory experiments were conducted to increase the skin penetration of the MBZ by determining the maximum solubility of MBZ in certain topical pharmaceutical skin penetrants as shown in Table 1 below. The maximum solubility of MBZ was tested in the various preferred solvent ingredients and combinations of ingredients, specifically, (1) dimethyl isosorbide (solvent composition 1), (2) diethylene glycol monoethyl ether (Transcutol P®) (solvent composition 2), (3) propylene glycol monolaurate (Lauryl Glycol®) (solvent composition 3), and (4) combinations of Transcutol P and Lauryl Glycol in a weight ratio of around 60:40 (solvent composition 4) were evaluated by adding 1% by weight of MBZ to each of them in the formulas listed in Table 1. All formulas were mixed at 20-25° C. for 24 hours with magnetic stirring followed by filtering through a 0.45 micron (u) membrane filter to remove undissolved solid particles of MBZ larger than 0.45 microns. Second batches of solvent compositions 1 (dimethyl isosorbide) and 2 (diethylene glycol monoethyl ether (Transcutol PR)) were also made and were further processed by exposure to a temperature of 100° C. for 15 minutes after filtering to form solvent compositions 5 and 6, respectively. All filtered formulas were crystal clear, except formulas 1 and 5 containing dimethyl isosorbide, which appeared milky white after filtration indicating solid MBZ with particle size less than 0.45 μ. This visual appearance indicates that solvent compositions 2-4 and 6 had better solubility than solvent compositions 1 and 5 (with dimethyl isosorbide or DMI) because undissolved, but small, solid particles remained in solvent compositions 1 and 5; however, each of the solvent compositions were assayed by UV-VIS spectrophotometric (300-325 nm) or by HPLC (Mebendazole; USP-NF Monograph) methods to determine the amount of MBZ in the compositions after the filtration step.

As shown in Table 1, the results of the two assay methods are in general agreement except for the solvent compositions 5 and 6 made with exposure to 100° C. temperature for 15 minutes, which show lower assay results with the HPLC assay indicating possible MBZ degradation due to the additional heating step.

TABLE 1
Saturation Solubility of MBZ in Solvent-Penetrants

| | UV-VIS Assay | HPLC Assay; |
|---|---|---|
| Solvent Composition | MBZ content | MBZ content |
| (with MBZ) | (ug/mL) | (ug) |
| 1 (Dimethyl Isosorbide)* | 5048 | 4870 |
| 2 (Transcutol P) | 2386 | 2390 |
| 3 (Propylene Glycol | 379 | 460 |
| Monolaurate) | | |
| 4 (Transcutol P/Propylene | 833 | 930 |
| Glycol Monolaurate | | |
| 60:40 w/w) | | |
| 5 (Dimethyl Isosorbide; | 4968 | 2700 |
| heated to 100° C. for 15 | | |
| minutes)* | | |
| 6 (Transcutol P; heated | 4188 | 3560 |
| 100° C. for 15 minutes) | | |

*Milky after filtration

The solubility of MBZ was found to be highest in either solvent composition 1 (DMI) or 2 (Transcutol P). However, this solubility is reduced to 10-20% of the theoretical maximum amount when the solvent compositions 1 and 2 are diluted 50% with deionized water. Although concentrations of up to 0.5% by weight MBZ can be achieved in undiluted solvent compositions containing (1) only MBZ and Transcutol P or (2) only MBZ and DMI, the resulting liquid solvent composition has undesirable cosmetic or aesthetic qualities, including a lingering oiliness on skin application. These issues can be avoided by diluting such solvent compositions with water (preferably deionized or purified water); however, the diluted solvent composition can be runny, making it difficult to apply to skin, particularly facial skin. These issues can be avoided with appropriate viscosity modifiers or gelling agents to create an MBZ treatment composition comprising a topical gel or thickened liquid. Such viscosity modifiers or gelling agents may be part of a topical application vehicle to which MBZ (or an MBZ concentrate composition) is added or may be part of an MBZ gel composition further described herein.

### Example 4—Mixed Solvent Composition Trials

Further laboratory experiments were conducted to evaluate a combination of Trancutol P and DMI as solvents for MBZ, to maximize MBZ solubility and skin penetration. DMI and Transcutol P were used at levels shown to be safe for application to human skin and diluted with deionized water to a concentration of 40% by weight Transcutol P and a concentration of 15% DMI for these trials, with the addition of 0.05-0.2% by weight MBZ, and the balance deionized water. Deionized water was used as a diluent in these trials, to effectively substitute for a topical application vehicle, such as a preferred embodiment of an aqueous MBZ cream base composition further described herein.

Experimentation with the blend of 40% w/w Transcutol P, 15% DMI and 43-44% deionized water and concentrations of MBZ at 0.05-0.2% by weight required heating of the solvent, water, and MBZ mixture to effect increased solubility of the MBZ. To keep temperatures as low as possible to avoid degradation of the MBZ, the solvent/water/MBZ mixtures were first stirred at 50° C. for 18 hours and then stirred at 60° C. for 18 hours. Test results showed some haziness of incomplete solubility and a color shift to yellow that was not observed with exposure of similar formulations heated to higher temperatures (90-100° C.) but for shorter times (5-15 minutes) such as compositions 5-6 in Example 3 (Table 1). Additionally, trials also showed that use of MBZ amounts higher than 0.15% by weight in the Transcutol P/DMI/deionized water (40/15/45% w/w) and MBZ solutions that were initially clear (indicating good solubility of the MBZ) could generate a more pronounced hazy precipitate after overnight storage. Based on these results, further formulation development testing on MBZ concentrations at 0.05-0.2% w/w and with heating at 60-90° C. for 5-15 minutes prior to adding the dilution water was conducted.

In these tests, it was found that the addition of heat to the MBZ/DMI/Transcutol P solvent mixture, prior to dilution with deionized water, solubility of MBZ increases in the final diluted composition or when mixed with an aqueous base vehicle, such as a preferred embodiment of an MBZ cream base composition herein. With heating between 60° C. and 90° C., increased solubility of MBZ at 0.05-0.15% by weight can be achieved with mixing for 5-15 minutes. At 60 and 70° C., initial complete solubility of MBZ is achieved at a concentration of 0.05% by weight. As the concentration is increased above 0.05% by weight, 0.10% w/w MBZ requires 80° C. and 0.15% w/w MBZ requires 90° C. for initial solubility. While one or more benzimidazoles, or specifically MBZ, may be initially completely dissolved in compositions herein, with further processing as described herein and/or with storage, some of the benzimidazoles or MBZ may precipitate out of solution, thereby making some compositions herein partially a solution and partially a suspension.

Thus, according to some embodiments, an MBZ concentrate composition is made by: (1) mixing an ethylene or propylene glycol based solvent (preferably diethylene glycol monoethyl ether (Transcutol PR)) with a sorbitol based solvent (preferably dimethyl isosorbide) and 0.05-0.2% w/w MBZ (more preferably 0.01-0.075% w/w MBZ); (2) heating to a temperature within a first temperature range; and (3) mixing or stirring during the heating step, such as by using a magnetic stirrer. Preferably, the first period of time is around 5 to 20 minutes, more preferably around 5 to 15 minutes. Most preferably, the heating temperature and first period of time will not result in degradation of the MBZ of more than 10%, more preferably not more than 8% and most preferably not more than 5%. The first temperature range is most preferably between at least 60° C. but less than a temperature at which MBZ will experience degradation of 5% or more. More preferably, the first temperature range is 60-90° C., and most preferably 70-90° C. Preferably, steps 1-3 are carried out before mixing the MBZ with any water or aqueous based application vehicle.

### Example 5—Trial Formulations—With and Without Microfluidization

In order to potentially increase the vehicle solubility potential and skin penetration of MBZ, MBZ concentrate compositions according to preferred embodiments were prepared and added to a topical MBZ cream base (or vehicle) formulation according to a preferred embodiment for testing (as further described in Example 6). Preferred embodiments of MBZ concentrate compositions for use in Example 6 are shown in Table 2. According to these embodiments, an MBZ solvent/skin penetrant concentrate composition (formula 45-147) was prepared as follows: (1) MBZ was micronized to a an average particle size of around 3.5 to 4.0 microns; (2) the micronized MBZ powder is added to a liquid mixture of (a) a sorbitol based solvent (preferably dimethyl isosorbide), (b) an ethylene and/or propylene glycol based solvent (preferably diethylene glycol monoethyl ether (Transcutol PR), propylene glycol monolaurate (Lauryl Glycol®) 60:40), and (c) a solubilizer and/or emulsifying agent (preferably Cremophor RH 40/polyoxyl 40 hydrogenated castor oil); and (3) the MBZ powder is mixed with the liquid ingredients preferably at 25-50° C., more preferably at 35-50° C., and for 15-30 minutes or until a uniform white suspension of MBZ powder in the liquid is created.

TABLE 2
Mebendazole Solvent and Penetrant Concentrate

| | | | Most |
|---|---|---|---|
| MBZ CONCENTRATE COMPOSITION (45-147) | | Preferred | Preferred |
| | TRIAL Ex. 6 | Range | Range |
| | PERCENT | PERCENT | PERCENT |
| INGREDIENTS | (w/w)* | (w/w) | (w/w) |
| Sorbitol based Solvent (DIMETHYL ISOSORBIDE) | 38.30 | 32.00-45.00 | 36.00-40.00 |
| MEBENDAZOLE USP | 3.60 | 1.00-10.00 | 3.00-6.00 |
| Ethylene or propylene glycol based solvent | 19.85 | 15.00-25.00 | 18.00-21.00 |
| (DIETHYLENE GLYCOL MONOETHYL ETHER | | | |
| (TRANSCUTOL P)) | | | |
| Solubilizer and/or emulsifying agent (POLYOXYL | 38.25 | 32.00-45.00 | 36.00-40.00 |
| 40 HYDROGENATED CASTOR OIL (CREMOPHOR RH 40)) | | | |
| TOTAL | 100.00 | | |

*Amounts of a preferred embodiment used in the MBZ concentrate composition used to prepare MBZ treatment compositions for Example 6 testing.

A laboratory batch of 1 Kg of MBZ concentrate composition (formula 45-147) was made according to the Trial Example 6 percentages in Table 2 and divided into substantially equal parts, some of which were microfluidized and some of which were not microfluidized, for use in preparing MBZ treatment compositions for use in Example 6. For the microfludized samples for use in Example 6, part of the MBZ concentrate batch is processed at 35-45° C. through a Microfluidics EH-110 microfluidizer (Microfluidics, Inc.) at 14,000 psi for 3 discreet complete passes. For the non-microfluidized samples, the MBZ concentrate batch is not processed further.

As an additional test for comparison of particle sizes, some of the MBZ concentrate batch was also microfluidized using 6 discreet passes through the microfluidizer under the same conditions described above for the 3-pass samples used for Example 6. The particle size of the MBZ concentrate batch samples that were not microfluidized (0 passes) and compared to the samples that were microfluidized after 3 passes and after 6 passes through the EH-110 microfluidizer was measured by instrumental laser diffraction by Particle Technology Laboratories (PTL, Downers Grove, IL) using a calibrated Malvern 3000 Mastersizer instrument. Table 3 summarizes the particle size related to undissolved and suspended solid MBZ. Average particle size for Dv(10), Dv(50), and Dv(90), which indicate the size median for which 10%, 50%, or 90%, respectively, of the particles within the distribution are smaller, are shown for each of the three samples. Four measurements were made for each of the three samples and an average of the four measurements is also shown in Table 3.

TABLE 3
Particle Size Analysis of 45-147 Concentrate with and without
Microfluidization

| | CUMULATIVE VOLUME % LESS THAN INDICATED SIZE {1, 1m) | | |
|---|---|---|---|
| SAMPLE ID | Dv (10) | Dv (50) | Dv (90) |

Sample 1: MBZ Topical Liquid Concentrate
Formula 45-147: 0 Passes (not microfluidized)

| | | | |
|---|---|---|---|
| Measurement 1 | 0.983 | 3.87 | 9.00 |
| Measurement 2 | 0.978 | 3.84 | 8.87 |
| Measurement 3 | 0.977 | 3.83 | 8.82 |
| Measurement 4 | 0.975 | 3.82 | 8.79 |
| Instrument Average | 0.978 | 3.84 | 8.87 |

Sample 2: MBZ Topical Liquid Concentrate
Formula 45-147; 3 Passes (microfluidized - 3 passes through the microfluidizer)

| | | | |
|---|---|---|---|
| Measurement 1 | 0.609 | 1.15 | 3.74 |
| Measurement 2 | 0.560 | 1.12 | 3.56 |
| Measurement 3 | 0.512 | 1.08 | 3.41 |
| Measurement 4 | 0.512 | 1.08 | 3.38 |
| Instrument Average | 0.544 | 1.11 | 3.52 |

Sample 3;MBZ Topical Liquid Concentrate
Formula 45-147; 6 Passes (microfluidized - 6 passes through the microfluidizer)

| | | | |
|---|---|---|---|
| Measurement 1 | 0.512 | 0.984 | 3.50 |
| Measurement 2 | 0.483 | 0.950 | 3.29 |
| Measurement 3 | 0.482 | 0.946 | 3.23 |
| Measurement 4 | 0.482 | 0.942 | 3.19 |
| Instrument Average | 0.489 | 0.956 | 3.30 |

As can be seen, the average Dv(50) of the zero pass Sample 1 (non-microfluidized) is 3.84 microns, which as expected is essentially the same as the Dv(50) of the micronized mebendazole compound powder used in the 45-147 formula. After 3 passes of 45-147 through the microfluidizer for Sample 2, 50% of all particles are smaller than 1.11 microns (the average Dv (50)). After 6 passes of 45-147 through the microfluidizer for Sample 3, 50% of all particles are smaller than 0.956 microns (the average Dv(50)). The average Dv(90) of 45-147 particles after 6 passes through the microfluidizer for Sample 3 shows 90% of all particles are less than 3.3 microns. The particle size analysis indicates that 3 or 6 discreet passes of 45-147 through the EH-110 microfluidizer substantially reduces the particle size distribution of undissolved MBZ as compared to the non-microfluidized sample. Additionally, 6 discreet passes achieves a larger reduction than 3 discreet passes. Reduction of the particle size may improve suspension uniformity over time in compositions according to embodiments herein in a suspension (or partial suspension) form and may improve MBZ skin penetration.

For compositions according to some embodiments herein where microfluidization is used, an MBZ concentrate composition may be microfluidized or a treatment composition (benzimidazoles/MBZ and a carrier) may be microfluidized. A method of microfluidization according to some embodiments comprises processing the composition to be microfluidized through a microfluidzer under one or more of the following conditions (1) a temperature in a range of 30-65° C., and more preferably 35-45° C. and/or (2) a pressure of 5,000-30,000 psi, more preferably 10,000-20,000 psi. A preferred microfluidzer is a Microfluidics EH-110 microfluidizer (commercially available from Microfluidics, Inc.), but other microfluidizers may also be used. The composition to be microfluidized may be passed through the microfluidizer for any number of discreet passes from 1 to 10 or more, but at least 3 passes is preferred. Alternatively, the composition may be continuously passed through a microfluidizer until a stable particle size distribution is achieved. Either way, the composition may be microfluidized until the desired particle size for undissolved, suspended benzimidazole/MBZ is achieved. For compositions utilizing micronized benzimidazoles/MBZ of an average particle size of 3.5 to 4.0 microns, microfluidization according to embodiments herein may achieve further reductions in undissolved benzimidazole/MBZ particle size in the composition, which may improve suspension uniformity over time in compositions according to embodiments herein in a suspension (or partial suspension) form and may improve MBZ skin penetration.

For use in Example 6, the 45-147 MBZ concentrate batches of zero pass microfluidization (i.e., not microfluidized) and 3-pass microfluidization as described above were then each mixed with an MBZ cream base (formula 45-149, according to the Trial Example 6 percentages in Table 4) in proportions that would create final MBZ treatment compositions containing 0.05%, 0.1%, 0.25%, 0.5% and 1% MBZ in the cream base vehicle (with ingredient amounts as shown in Table 5). This created a set of trial compositions that were microfluidized and a corresponding set (with the same ingredients/amounts) that were not microfluidized for testing in Example 6.

The topical MBZ cream base composition (also referred to as formulation 45-149) is an oil-in-water (O/W) type cream emulsion base vehicle, preferred embodiments of which are shown in Table 4.

TABLE 4
Mebendazole Vehicle Cream Base

| | | | Most |
|---|---|---|---|
| MBZ CREAM BASE 45-149 | | Preferred | Preferred |
| | TRIAL Ex. 6 | Range | Range |
| | PERCENT | PERCENT | PERCENT |
| INGREDIENTS | (w/w)* | (w/w) | (w/w) |
| WATER | 86.45 | 80.00-95.00 | 83.00-85.00 |
| GLYCERIN, USP | 2.00 | 1.00-4.00 | 1.50-2.50 |
| CARBOMER | 0.50 | 0.1-1.0 | 0.2-0.6 |
| DISODIUM EDTA | 0.05 | 0.02-0.20 | 0.05-0.1 |
| CETYL ALCOHOL | 2.00 | 1.00-4.00 | 1.50-2.50 |
| STEARYL ALCOHOL | 1.50 | 1.00-3.00 | 1.25-1.75 |
| GLYCERYL STERATE | 4.50 | 2.00-6.00 | 4.00-5.00 |
| (and) PEG 100 STEARATE | | | |
| POLYSORBATE 80 | 1.75 | 1.00-2.50 | 1.50-2.00 |
| TRIETHANOLAMINE 99% | 0.50 | 0.25-1.5 | 0.50-1.00 |
| PHENOXYETHANOL | 0.75 | 0.25-1.00 | 0.50-0.800 |
| TOTAL | 100.00 | | |

*Amounts of a preferred embodiment used in the MBZ Base Cream composition used to prepare MBZ treatment compositions for Example 6 testing.

The MBZ cream base preferably comprises an aqueous phase composition and an oil phase composition. The aqueous phase composition is preferably made as follows: (1) mix the glycerin and phenoxyethanol together until a clear uniform solution is created; (2) add the deionized water to the solution with mixing; (3) heat the aqueous mixture to 60-70° C., while continuing to mix/stir; (4) add the disodium EDTA with mixing until dissolved in the water phase; (5) slowly add the Carbomer 940 to the heated water phase by sprinkling on the water surface and mix for 45 minutes (maintaining temperature at 60-70° C.) until a uniform hydrated dispersion of the carbomer is created. The oil phase composition is preferably separately made as follows: (1) mix the (a) cetyl alcohol, (b) stearyl alcohol, (c) glyceryl stearate (and) PEG 100 stearate (which is an emulsifier composition), and (d) polysorbate 80 together; (2) heat the mixture to 60-70° C. with mixing until a uniform liquid oil phase is created. The MBZ cream base is preferably made by (1) adding the aqueous phase composition and the oil phase composition together; (2) heat to 60-70° C. with homogenization (Silverson L4RT-A homogenizer) at 5000-7000 rpm for 15-30 minutes; (3) cool to around 35-50° C., more preferably around 35° C., and add the triethanolamine while mixing until a smooth and uniform mixture is formed; and (4) further cool to around 25° C. (around room temperature). As further described below, it is most preferred that the MBZ concentrate composition be added and mixed with the MBZ cream base composition prior to cooling in step (4).

The final MBZ treatment compositions according to these embodiments were made by mixing the required amount of MBZ concentrate composition, previously heated to 35-50° C., with the required amount of MBZ cream base composition, also previously heated to 35-50° C., in the desired proportions, followed by cooling to room temperature. MBZ concentrate (45-147) not processed (“zero” passes) through an EH-110 microfluidizer and MBZ concentrate microfluidized (3 passes) as described previously were both used to create test formulations shown in Table 5.

Table 5 shows the amounts of ingredients for the MBZ cream base 45-149 as added to varying amounts of MBZ concentrate composition 45-147 to create a range of MBZ topical treatment compositions referred to herein as 45-151A through 45-151E having 0.05% up to 1% by weight MBZ.

TABLE 5
Mebendazole Treatment Compositions

| | 45-151 | 45-151 | 45-151 | 45-151 | 45-151 |
|---|---|---|---|---|---|
| | A | B | C | D | E |
| MBZ Treatment Compositions | | | | | |
| (Ex. 6 Test Formulations) | | | | | |
| MBZ Cream Base 45-149 Percent | 98.61 | 97.22 | 93.05 | 86.10 | 72.20 |
| MBZ Concentrate Phase 45-147 | 1.39 | 2.78 | 6.95 | 13.900 | 27.800 |
| Percent (microfluidilized or not | | | | | |
| microfluidized) | | | | | |
| Individual Ingredient Percentages | | | | | |
| in 45-151 MBZ Treatment Compositions | | | | | |
| WATER | 85.25 | 84.05 | 80.44 | 74.43 | 62.42 |
| GLYCERIN USP | 1.97 | 1.94 | 1.86 | 1.72 | 1.44 |
| CARBOMER | 0.49 | 0.49 | 0.47 | 0.43 | 0.36 |
| DISODIUM EDTA | 0.05 | 0.05 | 0.05 | 0.04 | 0.04 |
| CETYL ALCOHOL | 1.97 | 1.94 | 1.86 | 1.72 | 1.44 |
| STEARYL ALCOHOL | 1.48 | 1.46 | 1.40 | 1.29 | 1.08 |
| GLYCERYL STERATE (and) | 4.44 | 4.37 | 4.19 | 3.87 | 3.25 |
| PEG 100 STEARATE | | | | | |
| POLYSORBATE 80 | 1.73 | 1.70 | 1.63 | 1.51 | 1.26 |
| TRIETHANOLAMINE 99% | 0.49 | 0.49 | 0.47 | 0.43 | 0.36 |
| PHENOXYETHANOL | 0.74 | 0.73 | 0.70 | 0.65 | 0.54 |
| DIMETHYL ISOSORBIDE | 0.53 | 1.06 | 2.66 | 5.32 | 10.65 |
| MEBENDAZOLE, USP | 0.05 | 0.10 | 0.25 | 0.50 | 1.00 |
| DIETHYLENE GLYCOL MONOETHYL | 0.28 | 0.55 | 1.38 | 2.76 | 5.52 |
| ETHER (TRANSCUTOL P) | | | | | |
| POLYOXYL 40 HYDROGENATED | 0.53 | 1.06 | 2.66 | 5.32 | 10.63 |
| CASTOR OIL (CREMOPHOR RH 40) | | | | | |
| Total | 100.00 | 100.00 | 100.00 | 100.00 | 100.00 |

### Example 6—Skin PAMPA Testing for Permeation of MBZ With or Without Microfluidization

For use in Example 6, the MBZ treatment compositions 45-151A through 45-151E from Example 5 (Table 5) were created twice where one set of the treatment compositions used the first half of the lab batch of MBZ concentrate 45-147 that had been processed through the microfluidizer (3 passes) as previously described and a second set of the treatment compositions used the second half of the lab batch of MBZ concentrate 45-147 that had not been processed (zero passes) through the microfluidizer. As shown in Table 6, the microfluidized set is referred to with an added “M” (i.e., 45-151A-M through 45-151E-M) and the not microfluidized set is referred to without the added “M” (i.e., 45-151A through 45-151E). Other than the use of microfluidization or not, the treatment compositions in Example 6 correspond to those of the same letter in Example 5 (Table 5). For example, 45-151A-M is the same as 45-151A but the MBZ concentrate was processed through a microfluidizer in 45-151A-M. These sets of compositions, 45-151A-M through 45-151E-M and 45-151A through 45-151E, were used in testing in this Example 6.

MBZ treatment compositions 45-151A-M through 45-151E-M (with microfiluidization of the MBZ concentrate) and 45-151A through 45-151E (without microfluidization) were evaluated for skin penetration using the Skin PAMPA-Parallel Artificial Membrane Permeability Assay-in vitro artificial skin model (Pion, Inc.). Another treatment composition comprising Vanicream® plus 10% by weight MBZ as used in Example 2 was also tested as a control for comparison to the MBZ treatment compositions using MBZ concentrate compositions and MBZ cream base compositions according to some preferred embodiments herein. The Vanicream® plus 10% by weight MBZ (referred to treatment composition 45-151G) is also considered an MBZ treatment composition according to an embodiment of the disclosure, but for purposes of these tests was treated as the control. Table 6 lists the compositions tested in the Skin PAMPA skin model, where 150 microliters of each composition was applied to the Skin PAMPA artificial skin membrane and a PAMPA receptor (or acceptor media) fluid of 20% aqueous Hydroxypropyl-beta-cyclodextrin solution in a Prisma HT™ buffer. Prisma HT™ is a proprietary universal buffer concentrate formed by several compounds with pKa values evenly spaced to produce a constant buffer capacity in the range pH 3-10. The ionic strength of the PRISMA HTT is about 10 mM. For all trials, measurements were taken at 1, 6, and 24 hour intervals and the temperature was 32° C. For some of the trials, measurements were also taken at 0.25 and 0.5 hour intervals. In addition to the listed compositions, negative controls of the MBZ cream base (without MBZ) and Vanicream (without MBZ added) were also evaluated in the SKIN PAMPA tests to measure any interference in the UV assay of MBZ in the PAMPA acceptor plate solution. The UV absorbance of the negative control vehicles (Vanicream or MBZ cream base) was subtracted from the UV assay of their corresponding test compositions with MBZ. Table 6 lists the average permeated mass of MBZ through the artificial skin membrane, as well as the standard deviation, for each of the MBZ treatment compositions tested.

TABLE 6
Skin PAMPA Testing Compositions

| MBZ Treatment | | % API* |
|---|---|---|
| Composition | Process | (MBZ) |
| MBZ I.a 0.05% - | Microfluidized | 0.05% |
| 45-151A-M | | |
| MBZ I.a 0.1% - | | 0.1% |
| 45-151B-M | | |
| MBZ I.a 0.25% - | | 0.25% |
| 45-151C-M | | |
| MBZ I.a 0.5% - | | 0.5% |
| 45-151D-M | | |
| MBZ I.a 1% - | | 1% |
| 45-151E-M | | |
| MBZ II.a 0.05% - | Without | 0.05% |
| 45-151A | | |
| MBZ II.a 0.1% - | Microfluidization | 0.1% |
| 45-151B | | |
| MBZ II.a 0.25% - | | 0.25% |
| 45-151C | | |
| MBZ II.a 0.5% - | | 0.5% |
| 45-151D | | |
| MBZ II.a 1% - | | 1% |
| 45-151E | | |
| Vanicream plus 10% MBZ | N/A | 10% |

*% API is the percentage of active pharmaceutical ingredient (w/w)

Skin PAMPA Method Reference: Pharmaceutics 2021 Oct 21;13(11):1758. doi: 10.3390/pharmaceutics13111758.

TABLE 7A
Average Permeated Mass of Mebendazole at each time
point/SKIN PAMPA

| | Average Permeated Mass MBZ* [μg] | | | | | | | | | |
|---|---|---|---|---|---|---|---|---|---|---|
| MBZ Treatment | 0.25 hr | | 0.5 hr | | 1 hour | | 6 hours | | 24 hours | |
| Composition | Avg. | SD | Avg. | SD | Avg. | SD | Avg. | SD | Avg. | SD |
| MBZ I.a 0.05%- | 0.04 | 0.06 | 0.30 | 0.24 | 0.28 | 0.19 | 1.94 | 1.14 | 4.13 | 2.09 |
| 45-151A-M | | | | | | | | | | |
| MBZ I.a 0.1%- | 0.01 | 0.05 | 0.15 | 0.26 | 0.19 | 0.19 | 2.31 | 0.68 | 4.83 | 1.95 |
| 45-151B-M | | | | | | | | | | |
| MBZ I.a 0.25%- | 0.00 | 0.04 | 0.37 | 0.49 | 0.22 | 0.10 | 3.60 | 1.02 | 9.12 | 1.27 |
| 45-151C-M | | | | | | | | | | |
| MBZ I.a 0.5%- | 0.03 | 0.17 | 0.22 | 0.51 | 0.26 | 0.15 | 4.68 | 0.25 | 13.00 | 1.95 |
| 45-151D-M | | | | | | | | | | |
| MBZ I.a 1%- | −0.01 | 0.03 | 0.22 | 0.45 | 0.24 | 0.10 | 4.83 | 1.27 | 14.35 | 2.76 |
| 45-151E-M | | | | | | | | | | |
| MBZ II.a 0.05%- | −0.03 | 0.03 | 0.18 | 0.43 | 0.20 | 0.18 | 1.84 | 0.67 | 4.04 | 1.29 |
| 45-151A | | | | | | | | | | |
| MBZ II.a 0.1%- | −0.01 | 0.06 | 0.08 | 0.27 | 0.17 | 0,14 | 2.19 | 0.96 | 2.62 | 1.71 |
| 45-151B | | | | | | | | | | |
| MBZ II.a 0.25%- | 0.00 | 0.06 | 0.22 | 0.48 | 0.23 | 0.08 | 2.54 | 0.93 | 6.24 | 1.42 |
| 45-151C | | | | | | | | | | |
| MBZ II.a 0.5%- | 0.02 | 0.04 | 0.21 | 0.32 | 0.26 | 0.15 | 4.61 | 1.29 | 15.52 | 3.23 |
| 45-151D | | | | | | | | | | |
| MBZ II.a 1%- | 0.03 | 0.08 | −0.02 | 0.23 | 0.24 | 0.18 | 3.48 | 1.00 | 14.80 | 2.09 |
| 45-151E | | | | | | | | | | |
| Vanicream plus 10% | −0.03 | 0.05 | 0.23 | 0.56 | 0.44 | 0.18 | 5.02 | 1.75 | 17.14 | 4.74 |
| MBZ-45-151G | | | | | | | | | | |

*Total μg of MBZ in acceptor solution based on application of 150 μL MBZ Treatment Composition to 0.3 cm² membrane.

Table 7B lists total permeated mass of MBZ through the artificial skin membrane for each of the MBZ treatment compositions tested in Example 6.

TABLE 7B
Total Permeated Mass for Example 6

| | Total Permeated | | |
|---|---|---|---|
| | Amount* | Flux | Flux |
| | (24 hours) | (0-6 hours) | (6-24 hours) |
| MBZ Treatment Composition | [μg/cm²] | [μg/cm²/min] | [μg/cm²/min] |
| MBZ I.a 0.05% - | 22.3 | 0.024 | 0.013 |
| 45-151A-M | | | |
| MBZ I.a 0.1% - | 25.0 | 0.025 | 0.015 |
| 45-151B-M | | | |
| MBZ I.a 0.25% - | 44.37 | 0.039 | 0.028 |
| 45-151C-M | | | |
| MBZ I.a 0.5% - | 60.63 | 0.048 | 0.040 |
| 45-151DM | | | |
| MBZ I.a 1% - | 65.43 | 0.049 | 0.044 |
| 45-151E-M | | | |
| MBZ II.a 0.05% - | 20.77 | 0.00 | 0.012 |
| 45-151A | | | |
| MBZ II.a 0.1% - | 16.83 | 0.023 | 0.008 |
| 45-151B | | | |
| MBZ II.a 0.25% - | 30.77 | 0.028 | 0.019 |
| 45-151C | | | |
| MBZ II.a 0.5% - | 68.73 | 0.047 | 0.048 |
| 45-151D | | | |
| MBZ II.a 1% - | 61.77 | 0.035 | 0.046 |
| 45-151E | | | |
| Vanicream plus 10% MBZ - 45-151G | 75.98 | 0.024 | 0.013 |

*Total permeated amounts are recalculated to ug/cm² from the actual surface area of the Skin PAMPA membrane (0.3 cm²)

The summary results of the Skin PAMPA testing of the MBZ formulations in Example 6 indicate that permeation of mebendazole across the Skin PAMPA membrane from the 45-151 formulations is slow, and no mebendazole (or no appreciable mebendazole given the standard deviation) was observed in the acceptor plate at the 0.25, 0.5, and 1 hour time points. After 6 hours, mebendazole can be measured in the acceptor plate showing that it has permeated across the Skin PAMPA membrane, and a concentration dependency observed, with greater concentrations of sample within the vehicles resulting in a greater transmembrane flux.

At low treatment composition loading (lower concentrations or lower % API of MBZ of 0.05-0.25% MBZ), a slight increase in flux is observed in the microfluidization compositions (45-151A-M through 45-151E-M) compared with the compositions without microfluidization (45-151A through 45-151E). This indicates that the microfluidization process is beneficial in increasing dermal penetration at low concentrations of MBZ which may be simply related to greater MBZ dissolution. Low treatment composition loadings are also observed to undergo a significant reduction in the rate of MBZ flux after 6 hours which may be due to depletion of Mebendazole within the composition. Without being bound by theory, it is believed that this depletion is most likely due to a change in the flux dynamics—as the amount of MBZ saturates the membrane and enters the acceptor the concentration gradient of soluble and insoluble MBZ changes—rather than oxidation or other reaction causing a reduction in measurable MBZ. Similar trends are observed with a higher treatment composition loading (0.5-1%), but the changes in flux and permeated amount are less pronounced. Increasing the loading (increasing concentration or the % API of MBZ of 0.5-1% MBZ) in the treatment composition appears to negate these observed differences, with loadings greater than or equal to 0.5%, including the Vanicream®, showing similar permeation between vehicle types (MBZ cream base or Vanicream® and with or without microfluidization of the MBZ concentrate) and consistent flux at both short and long timescales.

Mebendazole flux and permeated amount is observed to increase with an increase of treatment composition loading, although permeated amount does not appear to increase proportionally with concentration of MBZ in the treatment compositions. Additionally, although the amounts using Vanicream® as the base vehicle for MBZ are slightly higher than when using the MBZ cream base as the base vehicle, no significant increase can be observed between the MBZ cream base vehicles containing 0.5% or 1% MBZ and the Vanicream plus 10% MBZ control. Given that the 45-151G Vanicream plus 10% MBZ control uses 10-20X the amount of MBZ compared to the 1% and 0.5% amounts in the treatment compositions using the MBZ concentrate (with its solvents designed to increase the solubility of the mebendazole and its penetration) in the MBZ cream base as the vehicle, there is a substantial improvement achieved through the use of the MBZ cream base according to preferred embodiments herein as they require substantially less active MBZ to achieve similar simulated dermal penetrations in the PAMPA model.

Since topical skin treatment of rosacea will most likely be chronic daily use with a topical MBZ treatment composition according to embodiments herein, the 6-hour and 24-hour cumulative penetration data for the Skin PAMPA test samples would seem to be most relevant. The 6-hour and 24-hour cumulative penetration of Mebendazole for the 45-151G Vanicream plus 10% MBZ control were not statistically different than the 0.5% and 1% MBZ treatment compositions 45-151E-M, 45-151D, and 45-151E with a range of approximately 12-21 ug of MBZ, indicating that the skin penetrants and MBZ solvents used in the MBZ concentrate composition 45-147 most likely were responsible for increasing the MBZ penetration relative to the MBZ content of the 45-151E-M, 45-151D, and 45-151E treatment compositions compared to the 45-151G Vanicream plus 10% MBZ control. The microfluidization processing used for the MBZ concentrate 45-147 and used in the 45-151D-M and 45-151E-M treatment compositions appears to have had no significant effect on MBZ penetration over the longer term 24 hour test. However, typically, the first 6 hours application is the more important interval for skin penetration and improvements were seen in that time frame for the microfluidized samples.

### Example 7—MBZ Concentrate Gel Compositions

For topical application to skin, it can be advantageous to have a thickened or gel composition containing MBZ. An MBZ gel composition is preferably one that can be directly applied to the skin with or without being pre-mixed with a separate vehicle or other vehicle ingredients, such as MBZ cream base compositions or Vanicream®. The use of the term “gel” is not intended to be limiting and includes thickened compositions, such as creams, ointments, suspensions, and emulsions, preferably having a viscosity of around 5,000 to 400,000 centipoise, more preferably of around 20,000 to 200,000 centipoise, that are suitable for application to human and/or animal skin.

MBZ gel compositions according to preferred embodiments include MBZ in combination with one or more of the other ingredients as shown in Table 8.

TABLE 8
Mebendazole Gel Compositions

| | Preferred | Most Preferred |
|---|---|---|
| | Range | Range |
| | PERCENT | PERCENT |
| MBZ Gel Composition Ingredients | (w/w) | (w/w) |
| MEBENDAZOLE USP | 0.01-0.5 | 0.05-0.15 |
| Sorbitol based Solvent (preferably DIMETHYL ISOSORBIDE) | 5-25 | 10-15 |
| Ethylene or propylene glycol based solvent (preferably | 15-50 | 25-45 |
| DIETHYLENE GLYCOL MONOETHYL ETHER | | |
| (TRANSCUTOL P)) | | |
| Solubilizer and/or emulsifying agent (POLYOXYL 40 | 0-10 | 0-5 |
| HYDROGENATED CASTOR OIL (CREMOPHOR RH 40)) | | |
| Cyclodextrin compound, preferably hydroxypropyl beta- | 0-15 | 5-9 |
| cyclodextrin (Cavasol ® W7 HP) | | |
| Viscosity modifying agent (preferably Hydroxyethylcellulose | 0.5-3 | 1-2 |
| (Natrosol ® 250HR)) | | |
| Water (preferably deionized) | 20-70 | 35-50 |

Table 9 below lists preferred embodiments of MBZ gel compositions 45-173A through 45-173F. According to one embodiment, these compositions were prepared and the at least partial dissolution of the MBZ in these compositions was achieved by: (1) moderate magnetic stirrer mixing of the Transcutol P and DMI and MBZ; (2) heating at the stated temperature (60, 70, 80, or 90° C.) for a first period of time of 5-15 minutes; (3) continuing to mix/stir during the heating step, such as by using the magnetic stirrer; and followed by (4) slow addition of water (preferably deionized), and any other ingredients (such as Natrosol gellant) at 35-50° C., while continuing to mix/stir to force cooling to room temperature within 15 minutes. To maximize solubility of the MBZ, it is important that steps (1)-(3) are carried out prior to the addition of water in step (4). According to other embodiments, one or more other ingredients, such as the viscosity modifying agents or gelling agents may be pre-mixed or pre-hydrated in the water prior to step (4).

TABLE 9
Mebendazole Gel Formulations/Heat Processing

| | 45-173-A | 45-173-B | 45-173-C | 45-173-D | 45-173-E | 45-173-F |
|---|---|---|---|---|---|---|
| Ingredient | (60° C.) | (70° C.) | (80° C.) | (90° C.) | (90° C.) | (90° C.) |
| Mebendazole, USP | 0.1 | 0.1 | 0.1 | 0.1 | 0.15 | 0.20 |
| Dimethyl Isosorbide | 15 | 15 | 15 | 15 | 15 | 15 |
| DIETHYLENE GLYCOL | 40 | 40 | 40 | 40 | 40 | 40 |
| MONOETHYL ETHER | | | | | | |
| (Transcutol P) | | | | | | |
| Hydroxyethylcellulose | 1 | 1 | 1 | 1 | 1 | 1 |
| (Natrosol ® 250HR) | | | | | | |
| Deionized Water | 43.9 | 43.9 | 43.9 | 43.9 | 43.85 | 43.8 |
| Total (% w/w) | 100 | 100 | 100 | 100 | 100 | 100 |

To prevent precipitation of dissolved MBZ from the compositions in Tables 2, 8 or 9, the addition of 2-10% of a cyclodextrin compound (preferably hydroxypropyl beta-cyclodextrin (Cavasol® W7 HP)) may optionally, but preferably, be added to the compositions. If the cyclodextrin compound is used, it is preferably first dissolved in the deionized water prior to the addition of the viscosity modifying agent (preferably hydroxyethylcellulose (Natrosol)). According to other embodiments, other viscosity modifying agents, such as carboxymethylcellulose, hydroxypropylcellulose, magnesium aluminum silicate, Carbomer, or other pharmaceutically acceptable viscosity modifying agents may be used in place of or in addition to hydroxyethylcellulose (Natrosol). Other ingredients that may improve or maintain stability of the compositions according to the disclosure, such as antioxidants, including ascorbic acid or ascorbate salts or ascorbyl esters, ferulic acid, ubidecarenone, dl-alpha tocopherol, BHT, BHA, or other pharmaceutically acceptable antioxidants may be added to the compositions according to some embodiments herein, as will be understood by those of ordinary skill in the art. Such antioxidant ingredients may be beneficial in improving or maintaining MBZ product stability.

### Example 8—Additional Skin PAMPA Testing for Permeation of MBZ With Transcutol P

Following the PAMPA study in Example 6, additional Skin PAMPA tests were conducted for Example 8. The purpose of Example 8 was to evaluate compositions that contained soluble mebendazole and minor amounts of insoluble suspended mebendazole at varying concentrations of total mebendazole in a topical gel treatment composition to try to achieve maximum penetration of mebendazole. Transcutol P is both a good solvent and penetrant for mebendazole and has been used in topical delivery systems at concentrations up to 45% by weight based on previous listing in the FDA Inactive Ingredient Database. In Example 6, combinations of Transcutol P and dimethylisosorbide (DMI) were evaluated; however, the solubility of mebendazole did not appear to be significantly enhanced with the DMI, so Transcutol P was used without DMI in Example 8.

Table 10 shows the amounts of ingredients for the MBZ topical treatment compositions 145-185A, 145-185C, and 145-185E, having 0.05% or 0.1% by weight MBZ along with Transcutol P, Natrasol® 250HX, and water. As with the prior skin PAMPA tests, composition 145-185G comprised Vanicream® plus 10% by weight MBZ as a control for comparison to the MBZ treatment compositions using MBZ with varying amounts of Transcutol P according to some embodiments of the disclosure. Additionally, several compositions with Transcutol P, Natrasol® 250HX, and water but no MBZ were also tested as controls, as was composition 145-185H which was Vanicream® without any MBZ. The primary purpose of these tests was to evaluate varying amounts of MBZ with varying amounts of Transcutol P, which was previously identified as both a good solvent for MBZ and a good skin penetrant. Dimethylisosorbide was not used in the treatment compositions in Example 8. The Vanicream® plus 10% by weight MBZ using in 145-185G is also considered an MBZ treatment composition according to an embodiment of the disclosure, but for purposes of these tests was treated as the control.

TABLE 10
Mebendazole Gel Treatment Compositions for Example 8 Testing

| MBZ Gel Composition Ingredients | 145-185A | 145-185C | 145-185E | 145-185G | 145-185H |
|---|---|---|---|---|---|
| (145-185 for Example 8) | (% w/w) | (% w/w) | (% w/w) | (% w/w) | (% w /w) |
| MEBENDAZOLE USP | 0.05 | 0.05 | 0.1 | 10.0 | N/A |
| Viscosity modifying agent (preferably | 1.0 | 1.0 | 1.0 | N/A | N/A |
| Hydroxyethylcellulose (Natrosol ® | | | | | |
| 250HX)) | | | | | |
| Ethylene or propylene glycol based | 40.0 | 20.0 | 40.0 | N/A | N/A |
| solvent (preferably DIETHYLENE | | | | | |
| GLYCOL MONOETHYL ETHER | | | | | |
| (TRANSCUTOL P)) | | | | | |
| Water (preferably deionized) | 58.95 | 78.95 | 58.9 | N/A | N/A |
| Vanicream ® | N/A | N/A | N/A | 90.0 | 100.00 |
| Total | 100.00 | 100.00 | 100.00 | 100.00 | 100.00 |

MBZ treatment compositions 145-185A, 145-185C, 145-185E and the control compositions were evaluated for skin penetration using the Skin PAMPA-Parallel Artificial Membrane Permeability Assay-in vitro artificial skin model (Pion, Inc.). A sample of 150 microliters of each composition was applied to the Skin PAMPA artificial skin membrane (donor plate) with a PAMPA receptor (or acceptor media) fluid of 20% aqueous Hydroxypropyl-beta-cyclodextrin solution in a Prisma buffer. This is the same acceptor media used in Example 6. Six replicates for each composition were tested. For all trials, measurements were taken at 0.25, 0.5, 1, 6, and 24 hour intervals and the temperature was 32° C. In addition to the listed compositions, a reference plate of known standards of MBZ with a 20% aqueous Hydroxypropyl-beta-cyclodextrin solution, with concentration ranges of 3.125-62.5 μg/mL were also evaluated in the SKIN PAMPA tests and the sum of absorbance within a wavelength range of 310-370 nm was used for sample quantitation for any interference in the UV assay of MBZ in the PAMPA acceptor plate solution. The UV absorbance of these known samples was subtracted from the UV assay of their corresponding test compositions with MBZ. Tables 11A-11B list the average permeated mass of MBZ through the artificial skin membrane, as well as the standard deviation, for each of the MBZ treatment compositions tested.

TABLE 11A
Average Permeated Mass of Mebendazole at each time
point/SKIN PAMPA for Example 8

| MBZ Treatment | Average Permeated Mass MBZ* [μg] | | | | | |
|---|---|---|---|---|---|---|
| Composition | 0.25 hr | | 0.5 hr | | 1 hour | |
| 145-185 | Avg. | SD | Avg. | SD | Avg. | SD |
| MBZ I.a 0.05%- | 0.061 | 0.047 | 0.838 | 1.008 | 0.005 | 0.005 |
| 145-185A | | | | | | |
| MBZ I.a 0.05%- | 0.010 | 0.016 | 1.964 | 1.483 | 0.002 | 0.004 |
| 145-185C | | | | | | |
| MBZ I.a 0.1%- | 0.008 | 0.013 | 0.073 | 0.178 | 0.007 | 0.007 |
| 145-185E | | | | | | |
| MBZ II.a 10%- | 0.003 | 0.006 | 1.271 | 1.817 | 0 | 0 |
| 145-185G | | | | | | |

*Total μg of MBZ in acceptor solution based on application of 150 μL MBZ Treatment Composition to 0.3 cm² membrane.

TABLE 11B
Average Permeated Mass of Mebendazole at each time point/SKIN PAMPA for Example 8

| MBZ Treatment | Average Permeated Mass MBZ* [μg] | | | |
|---|---|---|---|---|
| Composition | 6 hours | | 24 hours | |
| 145-185 | Avg. | SD | Avg. | SD |
| MBZ I.a 0.05% - | 0.373 | 0.207 | 0.809 | 0.786 |
| 145-185A | | | | |
| MBZ I.a 0.05% - | 0.426 | 0.260 | 2.518 | 1.200 |
| 145-185C | | | | |
| MBZ I.a 0.1% - | 0.290 | 0.230 | 2.742 | 1.66 |
| 145-185E | | | | |
| MBZ II.a 10% - | 0.329 | 0.134 | 3.225 | 1.380 |
| 145-185G | | | | |

*Total μg of MBZ in acceptor solution based on application of 150 μL MBZ Treatment Composition to 0.3 cm² membrane.

Table 11C lists total permeated mass of MBZ through the artificial skin membrane for each of the MBZ treatment compositions tested in Example 8.

TABLE 11C
Total Permeated Mass for Example 8

| | Total Permeated | | |
|---|---|---|---|
| MBZ Treatment | Amount* | Flux | Flux |
| Composition | (24 hours) | (0-6 hours) | (6-24 hours) |
| 145-185 | [μg/cm²] | [μg/cm²/min] | [μg/cm²/min] |
| MBZ I.a 0.05% - | 6.95 | 0.0118 | 0.0002 |
| 145-185A | | | |
| MBZ I.a 0.05% - | 16.4 | 0.0222 | 0.0004 |
| 145-185C | | | |
| MBZ I.a 0.1% - | 10.4 | 0.0035 | 0.0001 |
| 145-185E | | | |
| MBZ II.a 10% - | 16.09 | 0.0148 | 0.0001 |
| 145-185G | | | |

*Total permeated amount is recalculated and normalized for ug/cm² based on penetration results from the actual surface area of the Skin PAMPA membrane (0.3 cm²)

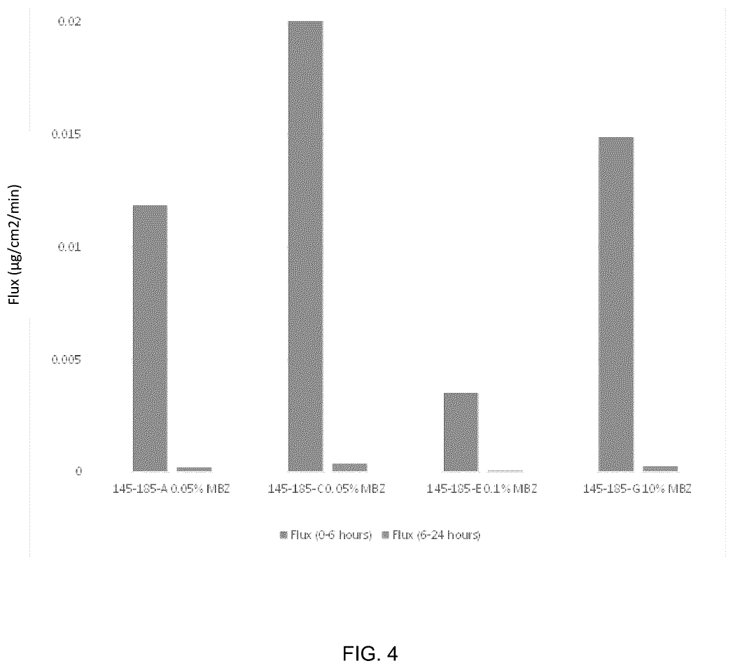

FIG. 4 is a graph depicting flux values at 0-6 hours compared to 6-24 hours for MBZ treatment compositions in Example 8.

The controls without MBZ had zero permeated MBZ, as expected. In Example 8, treatment composition 145-185C had the highest permeability across all time points, reaching a maximum normalized permeated amount of MBZ of 16.4 μg/cm², despite being the lowest concentration of MBZ (0.05%) and lowest concentration of Transcutol P (20%) of the treatment compositions tested. The second highest permeating formulation tested in this study was 145-185G, which is the Vanicream plus 10% MBZ treatment composition, reaching a maximum normalized permeated amount of MBZ of 16.1 μg/cm². Given the much higher concentration of MBZ in treatment composition 145-185G, a higher permeated amount is expected; however, the fact that treatment composition 145-185C had such a permeated amount (and even higher than 145-185G) with so much less MBZ in the treatment composition was unexpected.

As found in Example 6, the permeability of mebendazole through the PAMPA membrane is slow; however, unlike in Example 6, mebendazole in Example 8 can be observed in the acceptor plate at the 30 minute time point for treatment compositions 145-185A, 145-185C, 145-185E and 145-185G.

The treatment compositions in Example 8 also have a higher flux in the 0-6 hour time than the 6-24 hour time points. This is true for all four treatment compositions containing mebendazole (145-185A, 145-185C, 145-185E and 145-185G). This shows an improved absorbance in the relevant time point for a topical drug for these treatment compositions according to embodiments herein.

Interestingly, treatment composition 145-185G had a lower permeability than previously observed for the 45-151G Vanicream plus 10% MBZ treatment composition used in Example 6. Treatment composition 145-185G is materially the same as 45-151G except for a storage period between the time the tests for Examples 6 and 8 were conducted. The results in Example 8 show approximately four times lower cumulative permeated mass and flux than previously identified in Example 6. In Example 6, treatment composition 45-151G had 75.98 μg/cm² total permeated mass at 24 hours, with a flux of 0.024 μg/cm²/min at 0-6 hours and 0.013 μg/cm²/min. In Example 8, treatment composition 145-185 g had 16.09 μg/cm² total permeated mass at 24 hours, with a flux of 0.148 g/cm²/min at 0-6 hours and 0.01 μg/cm²/min at 6-24 hours. Without being bound by theory, these differences appear to due to changes in the UV active compounds in the Vanicream or in other structural changes in the Vanicream components or emulsion occurring between the time the tests for Example 6 were conducted and the tests for Example 8 and 9 were conducted, which could cause shifting in the base line spectra or permeation restrictions due to emulsion structural changes biasing results.

Additionally, as can be seen in Tables 11A-11C, treatment composition 145-185C comprising 20% Transcutol P and 0.05% MBZ had higher flux/penetration and total normalized 24 hr penetration (μg/cm2) than treatment composition 145-185E comprising 0.1% MBZ with 40% Transcutol P and treatment composition 145-185A comprising 0.05% MBZ and 40% Transcutol P. The ratio of Transcutol P to water for treatment composition 145-185C was approximately 1:4. This ratio appears to deliver a much higher flux and total penetration than formulations (1) at the same concentration of mebendazole and double the concentration of Transcutol P (0.05% MBZ and 40% Transcutol P in treatment composition 145-185A) or (2) double the concentration of both mebendazole and Transcutol P (0.1% MBZ and 40% Transcutol P in treatment composition 145-185E). Both treatment compositions 145-185A and 145-185E had ratios of Transcutol P to water of approximately 2:3.

### Example 9—Additional Skin PAMPA Testing With Different Receptor Fluid

In Example 9, additional Skin PAMPA tests were conducted on the same treatment compositions 145-185A, 145-185C, 145-185E, and 145-185G as used in Example 8 (see Table 10) using different receptor fluids (or acceptor media). The purpose of these tests was to determine if the mebendazole was accumulating in the simulated skin PAMPA membrane with the higher concentrations of Transcutol P used in treatment compositions 145-185A and 145-185E (both 40% Transcutol P) or possibly the higher concentration of Transcutol P was impeding the flux of mebendazole into the receptor fluid containing 20% hydroxypropyl beta-cyclodextrin (which was the solubilizer/receptor fluid used in Example 8).

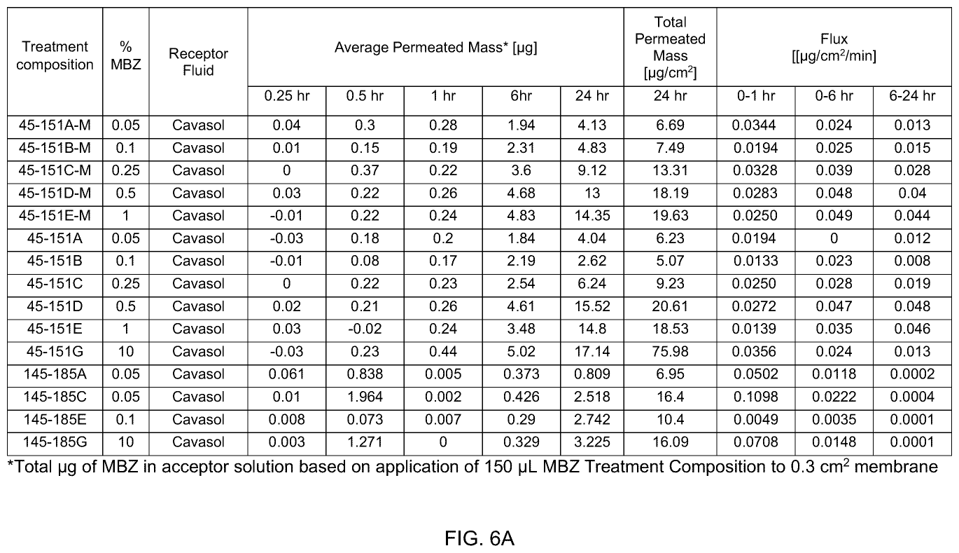

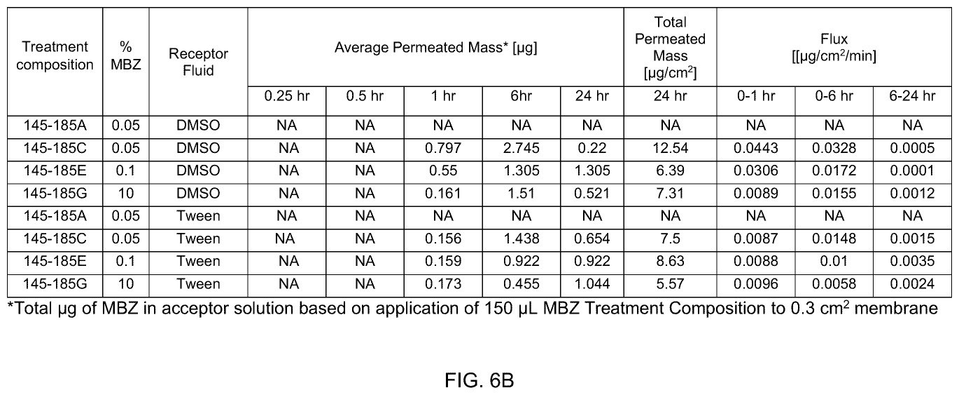

Example 9 repeated the testing of Example 8 for these treatment compositions with two new receptor fluids (or acceptor media) for comparison to the 20% hydroxypropyl beta-cyclodextrin (Cavasol®) used in Example 8. These two new receptor fluids were: (1) 20% dimethylsulfoxide (DMSO), and (2) 20% Polysorbate 80 (Tween 80) surfactant. Each receptor fluid also included Prisma buffer. Samples were taken at 1 hour, 6 hours, and 24 hours. The results of Example 9 were somewhat mixed. Table 12 lists total permeated mass of MBZ through the artificial skin membrane for each of MBZ treatment compositions tested with different receptor fluids. FIGS. 6A-6B are tables showing the data from Examples 6, 8, and 9 for comparison.

TABLE 12
Total Permeated Mass for Example 9

| | Total | | | |
|---|---|---|---|---|
| | Permeated | | | |
| | Amount* | Flux | Flux | Flux |
| MBZ Treatment Composition | (24 hours) | (0-1 hour) | (0-6 hours) | (6-24 hours) |
| 145-185 | [μg/cm²] | [μg/cm²/min] | [μg/cm²/min] | [μg/cm²/min] |
| MBZ I.a 0.05% MBZ | 16.4 | 0.1098 | 0.0222 | 0.0078 |
| 145-185C | | | | |
| 20% Cavasol | | | | |
| MBZ I.a 0.05% MBZ | 12.54 | 0.0443 | 0.0328 | 0.0005 |
| 145-185C | | | | |
| 20% DMSO | | | | |
| MBZ I.a 0.05% MBZ | 7.50 | 0.0087 | 0.0148 | 0.0015 |
| 145-185C | | | | |
| 20% Tween | | | | |
| MBZ I.a 0.1% MBZ | 10.4 | 0.0049 | 0.0035 | 0.0001 |
| 145-185E | | | | |
| 20% Cavasol | | | | |
| MBZ I.a 0.1% MBZ | 6.39 | 0.0306 | 0.0172 | 0.0001 |
| 145-185E | | | | |
| 20% DMSO | | | | |
| MBZ I.a 0.1% MBZ | 8.63 | 0.0088 | 0.0100 | 0.0035 |
| 145-185E | | | | |
| 20% Tween | | | | |
| MBZ II.a 10% MBZ | 16.09 | 0.0708 | 0.0148 | 0.0001 |
| 145-185G | | | | |
| 20% Cavasol | | | | |
| MBZ II.a 10% MBZ | 7.31 | 0.0089 | 0.0155 | 0.0012 |
| 145-185G | | | | |
| 20% DMSO | | | | |
| MBZ II.a 10% MBZ | 5.57 | 0.0096 | 0.0058 | 0.0024 |
| 145-185G | | | | |
| 20% Tween | | | | |

*Total permeated amount is recalculated and normalized for ug/cm² from actual amounts penetrating the surface area of the Skin PAMPA membrane (0.3 cm²)

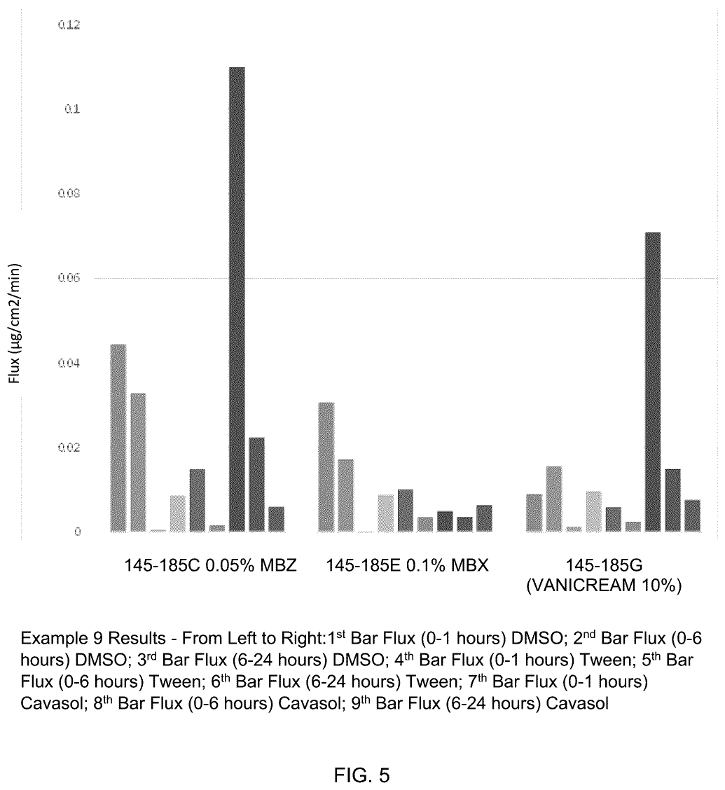

As can be seen from the data, each of treatment compositions 145-185C, 145-185-E, and 145-185G showed higher cumulative (total) permeated mass for the compositions in the initial PAMPA testing with 20% Cavasol compared to the 20% DMSO acceptor solutions at the 24-hour time point. For example, 145-185E reached 2.74 μg in Cavasol and 0.06 μg in DMSO, which is surprising given the solubility of MBZ in DMSO. However, treatment composition 145-185E showed a higher cumulative mass at the 6-hour time point in the DMSO (1.305 μg) compared to the Cavasol (0.29 μg), which performed the lowest at this time point. This is reflected in the flux data in FIG. 5 with the DMSO acceptor having a much greater flux at 0-1 hour and 0-6-hours time intervals compared to the Cavasol acceptor for treatment composition 145-185-E. Additionally, for treatment compositions 145-185C and 145-185G, total permeated mass at the 24-hour time point was higher in DMSO than in Tween, but was the opposite for treatment composition 145-185E.

Treatment compositions 45-151B-M (a cream with 0.1% MBZ with microfluidization) and 45-151B (a cream with 0.1% MBZ without microfluidization) from Example 6 can also be compared to the treatment compositions in Example 9. The full list of ingredients for these treatment compositions is included in Table 5 (for 45-151B), but each included 84.05% water, 1.06% dimethyl isosorbide, and 0.55% Transcutol P. Of these two treatment compositions, the highest cumulative permeated mass at 24 hours was the microfluidized treatment composition 45-151B-M with 25 μg/cm² whereas treatment composition 45-151B without microfluidization was 16.8 μg/cm². These were both in Cavasol as the receptor fluid. For comparison, 145-185E, which also had 0.1% MBZ without microfluidization and had a total permeated amount of MBZ after 24 hours of 10.4 μg/cm² in Cavasol.

Both treatment compositions 45-151B and 45-151B-M have a greater average permeated mass than treatment composition 145-185-E in Cavasol at the earlier 6-hour time point, at 2.19 μg, 2.31 μg, 0.29 μg, respectively. It should be noted that this is similar to the 0.05% formulations 145-185C, 45-151A-M and 45-151A in Cavasol for which the 6-hour time points are 0.426 μg, 1.94 μg, and 1.84 μg, respectively.

In the Tween acceptor solution, treatment composition 145-1850 reached the lowest total permeated mass (7.5 μg), with the 20% Cavasol performing the best (16.4 μg), after 24 hours.

The 45-151 series of MBZ cream formulations and the 10% MBZ in Vanicream contain a large excess of undissolved MBZ compared to the mostly solubilized MBZ with minor amounts of suspended MBZ in the 145-185 MBZ gel formulation series. Overall, the high amounts of undissolved and suspended MBZ in the 45-151 formula series results in relatively higher flux and MBZ permeation in the 6-24 hour time frame and relatively low flux and penetration in the 0-6 hour time frame. The 145-185 formulations, and particularly the 145-185 A and 145-185C (0.05% MBZ) formulations, showed higher flux and average permeated mass during the important 0-6 hr time frames for daily use topical facial product application.

As shown in FIG. 5, treatment composition 145-185C (0.05% MBZ) with a 20% Cavasol acceptor solution has a greater flux within the first hour (0.11 μg), compared to both treatment compositions 45-151A and 45-151B-M where flux results of 0.03 μg and 0.02 μg were found, respectively, also in the presence of a 20% Cavasol acceptor solution. A similar trend of greater flux for treatment composition 145-185C can be seen with the 20% DMSO acceptor solution. The increased 0-1 hour flux of treatment composition 145-185-C compared to the previous 0.05% MBZ treatment compositions may be significant for pharmacological activity due to the length of time it is practical for cream-based treatment compositions to be absorbed through a patient's skin. For example, the average permeated mass of MBZ in the first 30 minutes is nearly 10 times higher for 145-185C (Cavasol receptor) vs any of the 45-151 or 45-151-M series formulations at this early time point.

Treatment composition 145-185-E (0.1% MBZ) and using 20% Tween or 20% Cavasol shows a lower 0-1 hour, 0-6 hours and 6-24 hours flux compared to the 0.1% formulations in Example 6 (45-151B-M and 45-151B). The DMSO solution does appear to improve the 0-1 hour and 0-6 hours flux of treatment composition 145-185-E compared to the Cavasol acceptor. The improvement with 20% DMSO is not as great for the 0.1% mebendazole treatment composition 145-185E as compared to the 0.05% mebendazole treatment composition 145-185C. Without being bound by theory, this could occur due to the permeability of DMSO passing through the membrane and solubilizing the MBZ, whilst with the cyclodextrin only draws the already solubilized MBZ into the acceptor solution.

The results of the Vanicream® control treatment composition in Example 6 and Examples 8-9 are also unexpected. Generally, aside from the 0-1 hour flux result in 20% Cavasol in the acceptor for treatment composition 145-185G in Example 8, all of the flux values are lower for the Vanicream 10% treatment composition 145-185G in Example 9 than for the Vanicream 10% treatment composition in Example 6. Testing revealed evidence of differences in the UV spectra of the base cream used in these Examples. The calculation of sample concentration is taken from the sum of absorbance between 310-370 nm, which was previously determined to be a stable baseline for the calculation of UV activity for the compound. When comparing the average raw data from the Vanicream control (without MBZ) in Example 6 at 24 hours and the composition 145-185H from Example 8 (also a Vanicream control without MBZ), both with the same acceptor solution, it can be observed that there is a greater UV absorbance within the 310-370 nm wavelengths. As the Vanicream used in these Examples was from the same batch, this suggests some level of change in the base cream. The sum of absorbance of composition 145-185-H in Example 9 is 4 times higher than that of the Vanicream control in Example 6, which impacts on the calculation of the concentration and may be causing an over-correction of the 10% Vanicream in the acceptor solution in Example 9.

Table 14 lists the average permeated mass of MBZ through the artificial skin membrane, as well as the standard deviation, for each of the MBZ treatment compositions tested in Example 9. This data can be compared to Tables 11A-11B for these treatment composition in the Cavasol receptor fluid.

TABLE 14
Average Permeated Mass of Mebendazole at each time
point/SKIN PAMPA

| MBZ Treatment | Average Permeated Mass MBZ * [μg] | | | | | |
|---|---|---|---|---|---|---|
| Composition | 1 hour | | 6 hours | | 24 hours | |
| 145-185 | Avg. | SD | Avg. | SD | Avg. | SD |
| MBZ I.a 0.05% MBZ | 0.797 | 0.459 | 2.745 | 3.164 | 0.220 | 0.167 |
| 145-185C | | | | | | |
| 20% DMSO | | | | | | |
| MBZ I.a 0.1% MBZ | 0.550 | 0.038 | 1.305 | 1.063 | 0.060 | 0.077 |
| 145-185E | | | | | | |
| 20% DMSO | | | | | | |
| MBZ II.a 10% MBZ | 0.161 | 0.048 | 1.510 | 0.196 | 0.521 | 0.160 |
| 145-185G | | | | | | |
| 20% DMSO | | | | | | |
| MBZ I.a 0.05% MBZ | 0.156 | 0.148 | 1.438 | 0.586 | 0.654 | 0.378 |
| 145-185C | | | | | | |
| 20% Tween | | | | | | |
| MBZ I.a 0.1% MBZ | 0.159 | 0.049 | 0.922 | 1.058 | 1.51 | 2.158 |
| 145-185E | | | | | | |
| 20% Tween | | | | | | |
| MBZ II.a 10% MBZ | 0.173 | 0.085 | 0.455 | 0.313 | 1.044 | 0.746 |
| 145-185G | | | | | | |
| 20% Tween | | | | | | |

*Total μg of MBZ in acceptor solution based on application of 150 μL MBZ Treatment Composition to 0.3 cm² membrane.

From the results in Example 8, the 24-hour total mebendazole permeated amount was higher for the Ex. 145-185C treatment composition compared to both Exs. 145-185A and 145-185E treatment compositions with the cyclodextrin receptor. As can be seen in Example 9, the Ex. 145-185C treatment composition also had higher total 24-hour penetration amounts as compared to Ex. 145-185E with 20% DMSO as the acceptor. The 20% Tween receptor gave lower flux rates and lower total penetration than the cyclodextrin or the DMSO for all the test formulations but the lower flux rates for the Exs. 145-185C and 145-185E were comparable.

In addition to the surprising results that showed the 145-185-C treatment composition to deliver higher amount of mebendazole at lower concentrations of both the API (MBZ) and the Transcutol P solvent/penetrant, the 145-185-C treatment composition also delivers more mebendazole in a shorter period of time; i.e., a faster rate of mebendazole penetration. Tables 11A-11B show the flux rates or average permeated mass of mebendazole/time period for the 145-185C treatment composition in the Cavasol receptor fluid to be clearly higher than the 145-185A and 145-185E treatment compositions in the Cavasol receptor fluid during the 0-6 hour period. Similar faster and higher flux rates are also shown for the 145-185C treatment composition compared to the 145-185E treatment composition in Table 14 for 0-1 hour and 0-6 hour periods in the DMSO and Tween receptor fluids. These results indicate that treatment composition 145-185C according to an embodiment herein has a faster rate of penetration in each of the three different receptor fluid solvents tested-hydroxypropyl beta-cyclodextrin, DMSO, and Tween 80.

Statistical analysis of the penetration data for 145-185-A (0.05% MBZ/40% Transcutol) vs. 145-185-C (0.05% MBZ/20% Transcutol) and 145-185-C (0.05% MBZ/20% Transcutol) vs. 145-185-E (0.1% MBZ/40% Transcutol) was also conducted. Despite that these were small sample size runs (6 replicates), the penetration amount for 145-185-C (0.05% MBZ/20% Transcutol) was statistically greater than 145-185-A (0.05% MBZ/40% Transcutol) at the 95% confidence limit and 145-185-C (0.05% MBZ/20% Transcutol) penetration amount was greater than 145-185-E (0.1% MBZ/40% Transcutol) at the 90% confidence limit.

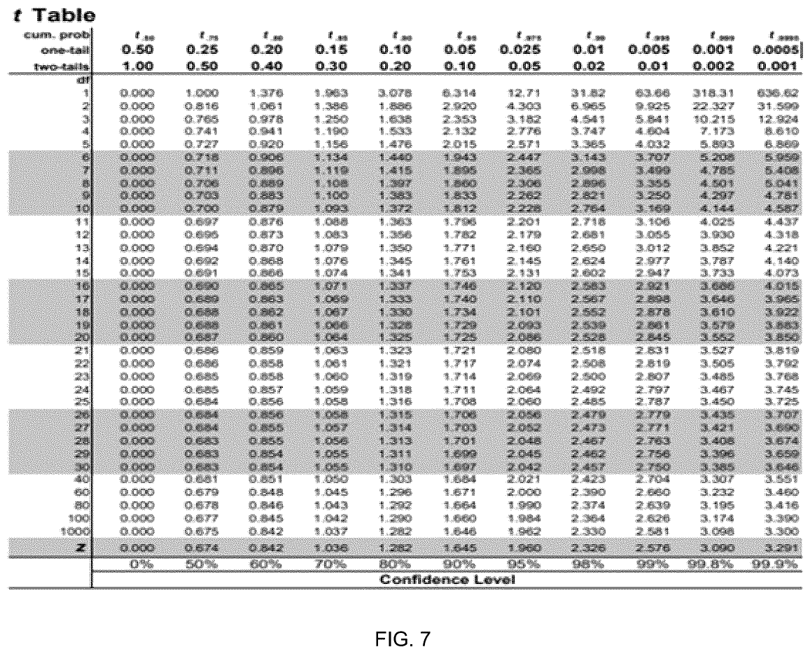

The t-stat results are shown for the comparison of 145-185-C to 145-185-A in Table 15 and for the comparison of 145-185-C to 145-185-E in Table 16 based on the total amount of permeated drug at the 24-hour time point and are shown in the tables below for the 95% confidence level. A more complete t-table is included in FIG. 7. The results show that 145-185C is significantly different compared to 145-185A at both the 90% and 95% confidence level. However, 145-185C is only significantly different to 145-185E at the 90% confidence level but it is not significantly different at the 95% confidence level.

TABLE 15
t-Stat Results for 145-185C Compared to 145-185A
20% cavasol
t-Test: Two-Sample Assuming Unequal Variances

| | 145-185-A 0.05% MBZ | 145-185-C 0.05% MBZ |
|---|---|---|
| | cavasol | cavasol |
| Mean | 2.085265685 | 4.920133963 |
| Variance | 1.885650594 | 2.730316361 |
| Observations | 6 | 6 |
| Hypothesized Mean | 0 | |
| Difference | | |
| df | 10 | |
| t Stat | −3.232043251 | |
| P(T <= t) one-tail | 0.004494888 | |
| t Critical one-tail | 1.812461123 | |
| P(T <= t) two-tail | 0.008989775 | |
| t Critical two-tail | 2.228138852 | |

TABLE 15
t-Stat Results for 145-185C Compared to 145-185E
C230086 20% cavasol
t-Test: Two-Sample Assuming Unequal Variances

| | 145-185-C 0.05% MBZ | 145-185-E 0.1% MBZ |
|---|---|---|
| | cavasol | cavasol |
| Mean | 4.920133963 | 3.120787631 |
| Variance | 2.730316361 | 2.155527943 |
| Observations | 6 | 6 |
| Hypothesized Mean | 0 | |
| Difference | | |
| df | 10 | |
| t Stat | 1.993978983 | |
| P(T <= t) one-tail | 0.037063924 | |
| t Critical one-tail | 1.812461123 | |
| P(T <= t) two-tail | 0.074127847 | |
| t Critical two-tail | 2.228138852 | |

Based on Examples 8-9, additional MBZ gel compositions according to preferred embodiments include MBZ in combination with one or more of the other ingredients as shown in Tables 17-18.

TABLE 17
Additional Mebendazole Gel Compositions

| | Preferred | Most Preferred |
|---|---|---|
| | Range | Range |
| | PERCENT | PERCENT |
| MBZ Gel Composition Ingredients | (w/w) | (w/w) |
| MEBENDAZOLE USP | 0.01-0.5 | 0.04-0.06 |
| Viscosity modifying agent (preferably Hydroxyethylcellulose | 0.5-3 | 0.75-1.25 |
| (Natrosol ® 250HX)) | | |
| Ethylene or propylene glycol based solvent (preferably | 15-45 | 15-25 |
| DIETHYLENE GLYCOL MONOETHYL ETHER (TRANSCUTOL | | |
| P)) | | |
| Water (preferably deionized) | 51.5-85.5 | 73.7-84.2 |

TABLE 18
Additional Mebendazole Gel Compositions

| | Preferred | Most Preferred |
|---|---|---|
| | Range | Range |
| | PERCENT | PERCENT |
| MBZ Gel Composition Ingredients | (w/w) | (w/w) |
| MEBENDAZOLE USP | 0.01-0.5 | 0.04-0.06 |
| Viscosity modifying agent (preferably Hydroxyethylcellulose | 0.5-3 | 0.75-1.25 |
| (Natrosol ® 250HX)) | | |
| Ethylene or propylene glycol based solvent (preferably | 15-45 | 15-25 |
| DIETHYLENE GLYCOL MONOETHYL ETHER (TRANSCUTOL | | |
| P)) | | |
| Cyclodextrin compound, preferably hydroxypropyl beta- | 0-15 | 5-9 |
| cyclodextrin (Cavasol ® W7 HP) | | |
| Water (preferably deionized) | 20-70 | 35-50 |

Various other approaches have been made and published to increase the water solubility and the GI absorption of mebenzadole's use as an anthelminthic. Any known methods or compositions for increasing water solubility and/or dermal absorption of mebendazole may also be used with compositions and methods according to embodiments herein.

According to still other embodiments, topical compositions, MBZ concentrate compositions, and MBZ treatment compositions comprise ingredients and amounts according to one or more of the following Embodiments:

Embodiment 1. An aqueous topical composition for treating or preventing an inflammatory or autoimmune disease or condition of human skin comprising 0.01 to 1% of one or more benzimidazole compounds and 15 to 45% of an ethylene glycol based solvent, the percentages by weight of the topical composition, wherein the one or more benzimidazole compounds are (1) fully dissolved in the aqueous topical composition, or (2) remain undissolved and suspended in the aqueous topical composition, or (3) partially dissolved in the aqueous topical composition and partially undissolved and suspended in the aqueous topical composition.

Embodiment 2. The topical composition of Embodiment 1 wherein the ethylene glycol based solvent comprises diethylene glycol monoethyl ether; and wherein the one or more benzimidazole compounds comprises mebendazole.

Embodiment 3. The topical composition of Embodiment 2 wherein topical composition is in a gel form and further comprises water and a viscosity modifying agent.

Embodiment 4. The topical composition of Embodiment 3 wherein the viscosity modifying agent comprises hydroxyethylcellulose.

Embodiment 5. The topical composition of Embodiment 4 comprising around 0.5 to 1.5% of the hydroxyehtylcellulose.

Embodiment 6. The topical composition of any one of Embodiments 1-5 wherein the one or more benzimidazole compounds comprises mebendazole and wherein the disease or condition comprises any form of rosacea.

Embodiment 7. The topical composition of any one of Embodiments 2-6 wherein the topical composition comprises: around 0.03 to 0.07% of the mebendazole; around 18 to 22% of the diethylene glycol monoethyl ether.

Embodiment 8. The topical composition of any one of Embodiments 1-7 further comprising a cyclodextrin compound.

Embodiment 9. The topical composition of Embodiment 8 wherein the cyclodextrin compound comprises hydroxypropyl beta-cyclodextrin.

Embodiment 10. The topical composition of Embodiment 9 comprising around 5 to 9% of the hydroxypropyl beta-cyclodextrin.

Embodiment 11. The topical composition of any one of Embodiments 1-10 wherein topical composition is in a gel form and further comprises water and a viscosity modifying agent.

Embodiment 12. The topical composition of Embodiment 11 wherein the viscosity modifying agent comprises hydroxyethylcellulose.

Embodiment 13. The topical composition of Embodiment 12 comprising around 0.5 to 1.5% of the hydroxyehtylcellulose.

Embodiment 14. A topical composition for treating or preventing an inflammatory or autoimmune disease or condition of human skin comprising 0.25 to 20% of one or more benzimidazole compounds, the percentage by weight of the topical composition.

Embodiment 15. The topical composition of Embodiment 14 further comprising a carrier suitable for application to human skin.

Embodiment 16. The topical composition of Embodiment 15 wherein the carrier comprises a cream, gel, lotion, liquid, emulsion, aerosol spray, non-aerosol spray, suspension, or ointment.

Embodiment 17. The topical composition of any one of Embodiments 15-16 wherein the carrier is aqueous.

Embodiment 18. The topical composition of any one of Embodiments 14-17 wherein the one or more benzimidazole compounds comprises mebendazole.

Embodiment 19. The topical composition of any one of Embodiments 14-18 wherein the disease or condition comprises any form of rosacea.

Embodiment 20. The topical composition of any one of Embodiments 14-19 wherein the one or more benzimidazole compounds comprises one or more of fenbenzadole, albenzadole, and thiabenzadole.

Embodiment 21 The topical composition of any one of Embodiments 14-20 wherein the topical composition comprises around 1-30% of a mebendazole concentrate composition and around 70-99% of vehicle composition suitable for application to human skin, the percentages by weight of the topical composition.

Embodiment 22. The topical composition of Embodiment 21 wherein the mebendazole concentrate composition comprises: around 1-10% mebendazole; around 32-45% of a sorbitol based solvent; around 15-25% of a glycol based solvent comprising an ethylene glycol based solvent, or a propylene glycol based solvent, or both; and around 32-45% total of a solubilizer or emulsifying agent or both; and wherein the percentages are by weight of the mebendazole concentrate composition.

Embodiment 23. The topical composition of Embodiment 22 wherein: the sorbitol based solvent comprises dimethyl isosorbide; the ethylene glycol based solvent comprises diethylene glycol monoethyl ether and the propylene glycol based solvent comprises propylene glycol monolaurate; and the solubilizer comprises polyoxyl 40 hydrogenated castor oil.

Embodiment 24. The topical composition of any one of Embodiments 14-23 wherein the topical composition comprises around 0.01 to 1% mebendazole by weight of the topical composition.

Embodiment 25. The topical composition of any one of Embodiments 14-24 wherein the topical composition has a viscosity of around 5000 to 400,000 centipoise.

Embodiment 26. The topical composition of any one of Embodiments 14-25 wherein the one or more benzimidazole compounds (or mebendazole) as an ingredient prior to any dissolution in the topical composition (or the mebendazole concentrate composition) comprise solid particles and wherein at least 50% of the solid particles have a particle size of around 3.5 to 4.0 microns or less.

Embodiment 27. An aqueous mebendazole treatment composition for topically treating or preventing an inflammatory or autoimmune disease or condition of human skin, the aqueous mebendazole treatment composition comprising: around 0.01-1% of one or more benzimidazole compounds; around 5-25% of a sorbitol based solvent; and/or around 15-50% of a glycol based solvent comprising an ethylene glycol based solvent, or a propylene glycol based solvent, or both; and wherein the percentages are by weight of the aqueous mebendazole treatment composition.

Embodiment 28. The aqueous mebendazole treatment composition of Embodiment 27 wherein: the one or more benzimidazole compounds comprises mebendazole; and/or the sorbitol based solvent comprises dimethyl isosorbide; and/or the ethylene glycol based solvent comprises diethylene glycol monoethyl ether; and/or the propylene glycol based solvent comprises propylene glycol monolaurate; and/or wherein the aqueous mebendazole composition has a viscosity of around 5,000 to 400,000 centipoise and/or wherein the 50% the mebendazole has a particle size of around 3.5 to 4.0 microns or less as an ingredient prior to any dissolution in the aqueous mebendazole composition.

Embodiment 29. The aqueous mebendazole treatment composition of any one of Embodiments 27-28 further comprising: around 1-10% total of a solubilizer or emulsifying agent or both; around 1-15% of a cyclodextrin compound; and/or around 0.5-3% of a viscosity modifying agent.

Embodiment 30. The aqueous mebendazole treatment composition of Embodiment 29 wherein: the solubilizer comprises polyoxyl 40 hydrogenated castor oil; the cyclodextrin compound comprises hydroxypropyl beta-cyclodextrin; and/or the viscosity modifying agent comprises hydroxyethylcellulose.

Embodiment 31. The aqueous mebendazole treatment composition of any one of Embodiments 27-30 wherein the composition is a gel.

Embodiment 32. The aqueous mebendazole treatment composition of any one of Embodiments 27-31 wherein the glycol based solvent is both the diethylene glycol monoethyl ether and the propylene glycol monolaurate in a weight ratio of around 55:45 to 65:35.

Embodiment 33. An aqueous topical suspension for treating or preventing an inflammatory or autoimmune disease or condition of human skin comprising: (1) 0.01 to 1% of one or more benzimidazole compounds, wherein at least some of the one or more benzimidazole compounds remain in undissolved form in the aqueous topical suspension; and (2) 15 to 45% of an ethylene glycol based solvent; wherein the percentages are by weight of the aqueous topical suspension; and wherein the aqueous topical suspension does not include any aprotic solvents.

Embodiment 34. The aqueous topical suspension of Embodiment 33 wherein the ethylene glycol based solvent comprises diethylene glycol monoethyl ether; and wherein the one or more benzimidazole compounds comprises mebendazole.

Embodiment 35. The aqueous topical suspension of any one of Embodiments 33-34 wherein aqueous topical suspension is in a gel form and further comprises water and a viscosity modifying agent.

Embodiment 36. The aqueous topical suspension of Embodiment 35 wherein the viscosity modifying agent comprises hydroxyethylcellulose.

Embodiment 37. The aqueous topical suspension of Embodiment 36 comprising around 0.5 to 1.5% of the hydroxyehtylcellulose.

Embodiment 38. The aqueous topical suspension of any one of Embodiments 33-37 wherein the one or more benzimidazole compounds comprises mebendazole.

Embodiment 39. The aqueous topical suspension of any one of Embodiments 34-38 wherein the aqueous topical suspension comprises: (1) around 0.03 to 0.07% of the mebendazole; and (2) around 18 to 22% of the diethylene glycol monoethyl ether.

Embodiment 40. The aqueous topical suspension of any one of Embodiments 33-39 further comprising a cyclodextrin compound.

Embodiment 41. The aqueous topical suspension of Embodiment 40 wherein the cyclodextrin compound comprises hydroxypropyl beta-cyclodextrin.

Embodiment 42. The aqueous topical suspension of Embodiment 41 comprising around 5 to 9% of the hydroxypropyl beta-cyclodextrin.

Embodiment 43. The aqueous topical suspension of any one of Embodiments 33-42 wherein aqueous topical suspension is in a gel form and further comprises water and a viscosity modifying agent.

Embodiment 44. The aqueous topical suspension of Embodiment 43 wherein the viscosity modifying agent comprises hydroxyethylcellulose.

Embodiment 45. The aqueous topical suspension of Embodiment 44 comprising around 0.5 to 1.5% of the hydroxyehtylcellulose.

Embodiment 46. The aqueous topical suspension of any one of Embodiments 34-45 wherein the topical composition comprises: around 0.03 to 0.07% of the mebendazole; around 18 to 22% of the diethylene glycol monoethyl ether.

Embodiment 47. The aqueous topical suspension of any one of Embodiments 34-46 wherein the one or more benzimidazole compounds comprises one or more of fenbenzadole, albenzadole, and thiabenzadole.

Embodiment 48. The aqueous topical suspension of any one of Embodiments 34-47 wherein the topical composition comprises around 1-30% of a mebendazole concentrate composition and around 70-99% of vehicle composition suitable for application to human skin, the percentages by weight of the aqueous topical suspension.

Embodiment 49. The aqueous topical suspension of Embodiment 48 wherein the mebendazole concentrate composition comprises: around 1-10% mebendazole; around 32-45% of a sorbitol based solvent; around 15-25% of a glycol based solvent comprising an ethylene glycol based solvent, or a propylene glycol based solvent, or both; and around 32-45% total of a solubilizer or emulsifying agent or both; and wherein the percentages are by weight of the mebendazole concentrate composition.

Embodiment 50. The aqueous topical suspension of Embodiment 49 wherein: the sorbitol based solvent comprises dimethyl isosorbide; the ethylene glycol based solvent comprises diethylene glycol monoethyl ether and the propylene glycol based solvent comprises propylene glycol monolaurate; and the solubilizer comprises polyoxyl 40 hydrogenated castor oil.

Embodiment 51. The aqueous topical suspension of any one of Embodiments 33-50 wherein the aqueous topical suspension has a viscosity of around 5000 to 400,000 centipoise.

Embodiment 52. The aqueous topical suspension of any one of Embodiments 33-51 wherein the one or more benzimidazole compounds as an ingredient prior to any dissolution in the aqueous topical suspension (or the mebendazole concentrate composition) comprise solid particles and wherein at least 50% of the solid particles have a particle size of around 3.5 to 4.0 microns or less.

According to still other preferred embodiments, a method of treating or preventing an inflammatory or autoimmune disease or condition of human or animal skin comprises steps, ingredients, and amounts according to one or more of the following Embodiments:

Embodiment 53. A method for treating or preventing an inflammatory or autoimmune disease or condition of human skin, the method comprising applying a topical composition comprising a carrier and 0.01 to 20% by weight of one or more benzimidazole compounds to an area of a subject's skin affected by the disease or condition.

Embodiment 54. The method of Embodiment 53 wherein the carrier is aqueous.

Embodiment 55. The method of any one of Embodiments 53-54 wherein the carrier comprises a cream, gel, lotion, liquid, emulsion, aerosol spray, non-aerosol spray, suspension, or ointment for topical application to skin.

Embodiment 56. The method of any one of Embodiments 53-55 wherein the one or more benzimidazole compounds comprises mebendazole

Embodiment 57. The method of any one of Embodiments 53-56 wherein the disease or condition comprises any form of rosacea.

Embodiment 58. The method of any one of Embodiments 52-57 wherein the applying step is repeated at least once per day for a treatment period comprising at least two weeks.

Embodiment 59. The method of any one of Embodiments 53-58 wherein the applying step comprises applying around 0.025 to 0.5 g to each side of the facial skin affected by rosacea of the topical composition.

Embodiment 60. The method of any one of Embodiments 53-59 wherein the method achieves a reduction in the number of cutaneous cytotoxic CD+8 T-cells of 50% or greater in the area of skin at the end of the treatment period compared to a number of cutaneous cytotoxic CD+8 T-cells in the area prior to the treatment period.

Embodiment 61. The method of any one of Embodiments 53-60 wherein the method achieves a reduction of a number of papules or pustules or diffuse redness intensity of 25% or greater on the area of skin at the end of the treatment period compared a number of papules or pustules on the area prior to the treatment period.

Embodiment 62. The method of any one of Embodiments 53-61 further comprising repeating the applying step at least once per day for a treatment period comprising at least twelve weeks; wherein the one or more benzimidazole compounds comprises mebendazole; wherein the carrier comprises a cream, gel, lotion, liquid, emulsion, aerosol spray, non-aerosol spray, suspension, or ointment for topical application to skin; wherein the disease or condition comprises any form of rosacea; and wherein the method achieves a reduction in the number of cutaneous cytotoxic CD+8 T-cells of 50% or greater in the area of skin at the end of the treatment period compared to a number of cutaneous cytotoxic CD+8 T-cells in the area prior to the treatment period.

Embodiment 63. The method of any one of Embodiments 53-62 wherein the topical composition is according to any one of Embodiments 1-13.

Embodiment 64. The method of any one of Embodiments 532-62 wherein the topical composition is according to any one of Embodiments 14-26.

Embodiment 65. The method of any one of Embodiments 53-62 wherein the topical composition is the aqueous mebendazole treatment composition according to any one of Embodiments 27-32.

Embodiment 66. The method of any one of Embodiments 53-62 wherein the topical composition is the aqueous topical suspension according to any one of Embodiments 33-52.

Embodiment 67. A method for treating or preventing an inflammatory or autoimmune disease or condition of human skin, the method comprising applying a topical composition comprising to an area of human skin affected by the disease or condition; wherein the topical composition comprises an aqueous carrier and 0.01 to 1% by weight of one or more benzimidazole compounds; and wherein the aqueous topical suspension does not include any aprotic solvents.

Embodiment 68. The method of Embodiment 67 wherein the topical composition is in a suspension form wherein at least some of the one or more benzimidazole compounds are in undissolved form suspended in the topical composition.

Embodiment 69. The method of any one of Embodiments 67-68 wherein the one or more benzimidazole compounds comprises mebendazole and wherein the disease or condition comprises any form of rosacea.

Embodiment 70. The method of any one of Embodiments 67-69 wherein applying the topical composition is repeated at least once per day for a treatment period comprising at least two weeks; and wherein applying the topical composition comprises applying around 0.025 to 0.5 g to the area of human skin; and wherein the area of human skin comprises facial skin.

Embodiment 71. The method of any one of Embodiments 67-70 wherein the method achieves a reduction in the number of cutaneous cytotoxic CD+8 T-cells of 50% or greater in the area of human skin at an end of the treatment period compared to a number of cutaneous cytotoxic CD+8 T-cells in the area of human skin prior to the treatment period.

Embodiment 72. The method of any one of Embodiments 67-71 wherein the topical composition is according to any one of Embodiments 1-13.

Embodiment 73. The method of any one of Embodiments 67-71 wherein the topical composition is according to any one of Embodiments 14-26.

Embodiment 74. The method of any one of Embodiments 67-71 wherein the topical composition is the aqueous mebendazole treatment composition according to any one of Embodiments 27-32.

Embodiment 75. The method of any one of Embodiments 67-71 wherein the topical composition is the aqueous topical suspension according to any one of Embodiments 33-52.

According to still other preferred embodiments, a method of making a benzimidazole treatment composition, or a mebendazole treatment composition, for topically treating or preventing an inflammatory or autoimmune disease or condition of human skin comprises steps, ingredients, and amounts according to one or more of the following paragraphs:

Embodiment 76. A method of making a benzimidazole treatment composition for topically treating or preventing an inflammatory or autoimmune disease or condition of human skin, the method comprising: (1) adding an amount of one or more solvents and an amount of one or more benzimidazole compounds to form a first mixture; and (2) heating the first mixture to a first temperature within a first temperature range for a first period of time while mixing or stirring to form a heated mixture; and wherein the amount of the one or more benzimidazole compounds is around 0.01-1.0, preferably around 0.01-0.2%, more preferably 0.01-0.075%, by weight of the benzimidazole treatment composition.

Embodiment 77. The method of Embodiment 76 wherein the first period of time is around 5 to 20 minutes, more preferably around 5 to 15 minutes; and/or wherein the first temperature range is at least 60° C. but less than a temperature at which the one or more benzimidazole compounds will experience degradation of 5% or more.

Embodiment 78. The method of any one of Embodiments 76-77 wherein the amount of mebendazole is 0.05-0.075%.

Embodiment 79. The method of any one of Embodiments 76-78 wherein the first temperature range is 60-90° C.

Embodiment 80. The method of any one of Embodiments 76-78 wherein the first temperature range is 70-100° C., more preferably 70-90° C.

Embodiment 81. The method of any one of Embodiments 76-80 wherein the first period of time is 5 to 15 minutes.

Embodiment 82. The method of any one of Embodiments 76-81 wherein the one or more solvents comprise a sorbitol based solvent, or a glycol based solvent or both.

Embodiment 83. The method of Embodiment 82 wherein: (1) the sorbitol based solvent comprises dimethyl isosorbide; and/or (2) the glycol based solvent comprises diethylene glycol monoethyl ether, propylene glycol monolaurate, or both.

Embodiment 84. The method of any one of Embodiments 76-83 further comprising: cooling the heated composition to a second temperature within a second range of temperatures by adding water and mixing or stirring to form a cooled composition; and wherein the second range of temperatures is around 35-50° C.

Embodiment 85. The method of Embodiment 84 wherein the cooling the heated mixture is completed in 15 minutes or less.

Embodiment 86. The method of any one of Embodiments 84-85 further comprising dissolving an amount of a viscosity modifying agent or an amount of an anti-precipitation agent or both in the water prior to the cooling step.

Embodiment 87. The method of Embodiment 86 wherein the viscosity modifying agent comprises hydroxyethylcellulose.

Embodiment 88. The method of any one of Embodiments 86-87 wherein the anti-precipitation agent comprises a cyclodextrin compound and the amount of the anti-precipitation agent is around 2-10% by weight of the mebendazole treatment composition.

Embodiment 89. The method of Embodiment 88 wherein cyclodextrin compound comprises hydroxypropyl beta-cyclodextrin.

Embodiment 90. The method of any one of Embodiments 86-89 wherein the heated composition or the cooled composition or the mebendazole treatment composition is microfluidized in a microfluidizer at 5,000-30,000 psi for at least one pass.

Embodiment 91. The method of Embodiment 90 wherein the heated composition or the cooled composition or the mebendazole treatment composition is microfluidized in a microfluidizer at 5,000-20,000 psi for at least three passes.

Embodiment 92. The method of any one of Embodiments 86-89 wherein the method does not comprise a microfluidization step.

Embodiment 93. The method of any one of Embodiments 76-88 wherein the heated composition or the benzimidazole treatment composition is microfluidized in a microfluidizer at 5,000-30,000 psi for at least one pass.

Embodiment 94. The method of Embodiment 93 wherein the heated composition or the benzimidazole treatment composition is microfluidized in a microfluidizer at 5,000-20,000 psi for at least three passes.

Embodiment 95. The method of any one of Embodiments 76-94 wherein the benzimidazole treatment composition has a viscosity of around 5,000 to 400,000 centipoise.

Embodiment 96. The method of any one of Embodiments 76-95 wherein the one or more benzimidazole compounds as an ingredient prior to adding and mixing with the one or more solvents comprises solid particle, the method further comprising micronizing the one or more benzimidazole compounds so that at least 50% of the solid particles have a particle size of around 3.5 to 4.0 microns prior to the adding step.

Embodiment 97. The method of any one of Embodiments 76, 78, or 82-96 wherein the first temperature range is 25-50° C. and the first time period is 15-30 minutes.

Embodiment 98. The method of any one of Embodiments 76-97 wherein at least some of the one or more benzimidazole compounds remains in undissolved form and suspended in the mebendazole treatment composition.

Embodiment 99. The method of any one of Embodiments 76-98 wherein the benzimidazole treatment composition does not include any aprotic solvents.

Embodiment 100. The method of any one of Embodiments 76-99 wherein the one or more benzimidazole compounds comprises one or more of fenbenzadole, albenzadole, and thiabenzadole.

Embodiment 101. The method of any one of Embodiments 76-99 wherein the one or more benzimidazole compounds comprises mebendazole.

Embodiment 102. The method of any one of Embodiments 76-99 wherein the benzimidazole treat composition is according to any one of Embodiments 1-13.

Embodiment 103. The method of any one of Embodiments 76-99 wherein the benzimidazole treat composition is according to any one of Embodiments 14-26.

Embodiment 104. The method of any one of Embodiments 76-99 wherein the benzimidazole treat composition is the aqueous mebendazole treatment composition according to any one of Embodiments 27-32.

Embodiment 105. The method of any one of Embodiments 76-99 wherein the benzimidazole treatment composition is the aqueous topical suspension according to any one of Embodiments 33-52.

Embodiments of compositions and methods of the disclosure are capable of achieving one or more of the following benefits: (1) reducing T-lymphocyte cell density by at least around 50%, more preferably by at least around 90% compared to before treatment with a mebendazole composition; (2) reducing the number of papules and/or pustules or diffuse redness on the skin of a rosacea patient by at least around 25%, more preferably by at least around 75% compared to before treatment with a mebendazole composition; (3) reducing the appearance of swelling on the skin compared to before treatment with a mebendazole composition; (4) reducing a level of itchiness, or hot or burning sensation on the skin compared to before treatment with a mebendazole composition; and/or (5) for the treatment of erythematotelangiectactic rosacea, a significant reduction in the redness “a” or “a*” value of the facial skin is demonstrated with the topical mebenzadole treatment. The “a” or “a*” value is an objective measurement of redness as measured by the Hunter L,a,b (Hunter Labs) and CIE L*a*b* (CIELAB) colorimeters, the Minolta CR type colorimeters or other suitable colorimeter or chromameter. Visible changes in redness can be detected in colorimeter a* units as low as 0.2 units and a decrease in one (1.0) or more a or a* colorimeter units after topical treatment of erythematotelangiectactic rosacea is generally considered to show clinical efficacy.

Embodiments of MBZ treatment compositions and methods according to the disclosure are also capable of achieving cumulative skin penetrations after 24 hours of at least 5 ug of MBZ, more preferably at least 10 μg, most preferably at least 15 μg. These amounts are as measured in acceptor solution using the Skin PAMPA tests described herein and based on application of 150 μL MBZ Treatment Composition to 0.3 cm² artificial skin membrane.

All numerical values, ratios, or percentages indicated herein as a range include each individual amount, numerical value, or ratio within those ranges and any and all subset combinations within ranges, including subsets that overlap from one preferred range to a more preferred range. References to “about” or “around” with respect to numerical values generally mean (1) +/−1 for values expressed as whole numbers (without a decimal, e.g., around 15% means 14-16%); (2) +/−0.1 for values expressed with a single decimal place (for example, around 9.5% means 9.4-9.6%; and (3) +/−0.01 for values expressed with two or more decimal places (for example, around 0.02 means 0.01-0.03, each of the foregoing excluding values that would result in a negative number. Any ingredient other than the benzimidazole described as included in an embodiment herein may also be excluded from such embodiment. Unless specifically excluded, any ingredients, preferred features, and/or optional ingredients any treatment composition embodiment and/or method steps described herein may be used with any other embodiment, even if not specifically described herein with that particular embodiment. Any treatment composition embodiment herein may comprise, consist essentially of, or consist of any combination of ingredients described herein. References herein to water (without any modifier) include potable water, distilled water, deionized water, or other forms of purified, filtered, or cleaned water suitable for use in topical skin treatment compositions. These forms of water may be substituted for references herein to deionized water, other than in the claims.

Those of ordinary skill in the art will also appreciate upon reading this specification, including the examples contained herein, that modifications and alterations to the preferred embodiments of a composition and its method of use may be made within the scope of the disclosure and it is intended that the scope of the invention disclosed herein be limited only by the broadest interpretation of the appended claims to which the inventor is legally entitled.

We claim:

1. An aqueous topical suspension for treating or preventing an inflammatory or autoimmune disease or condition of human skin comprising:
0.01 to 1% of one or more benzimidazole compounds, wherein at least some of the one or more benzimidazole compounds remain in undissolved form in the aqueous topical suspension; and
15 to 45% of an ethylene glycol based solvent;
wherein the percentages are by weight of the aqueous topical suspension; and
wherein the aqueous topical suspension does not include any aprotic solvents.

2. The aqueous topical suspension of claim 1 wherein the ethylene glycol based solvent comprises diethylene glycol monoethyl ether; and
wherein the one or more benzimidazole compounds comprises mebendazole.

3. The aqueous topical suspension of claim 2 wherein aqueous topical suspension is in a gel form and further comprises water and a viscosity modifying agent.

4. The aqueous topical suspension of claim 3 wherein the viscosity modifying agent comprises hydroxyethylcellulose.

5. The aqueous topical suspension of claim 4 comprising around 0.5 to 1.5% of the hydroxyehtylcellulose.

6. The aqueous topical suspension of claim 1 wherein the one or more benzimidazole compounds comprises mebendazole.

7. The aqueous topical suspension of claim 2 wherein the aqueous topical suspension comprises:
around 0.03 to 0.07% of the mebendazole;
around 18 to 22% of the diethylene glycol monoethyl ether.

8. The aqueous topical suspension of claim 7 further comprising a cyclodextrin compound.

9. The aqueous topical suspension of claim 8 wherein the cyclodextrin compound comprises hydroxypropyl beta-cyclodextrin.

10. The aqueous topical suspension of claim 9 comprising around 5 to 9% of the hydroxypropyl beta-cyclodextrin.

11. The aqueous topical suspension of claim 10 wherein the aqueous topical suspension is in a gel form and further comprises water and a viscosity modifying agent.

12. The aqueous topical suspension of claim 11 wherein the viscosity modifying agent comprises hydroxyethylcellulose.

13. The aqueous topical suspension of claim 12 comprising around 0.5 to 1.5% of the hydroxyehtylcellulose.

14. A method for treating or preventing an inflammatory or autoimmune disease or condition of human skin, the method comprising applying a topical composition comprising to an area of human skin affected by the inflammatory or autoimmune disease or condition;
wherein the topical composition comprises an aqueous carrier and 0.01 to 1% by weight of one or more benzimidazole compounds; and
wherein the topical composition does not include any aprotic solvents.

15. The method of claim 14 wherein the topical composition is in a suspension form wherein at least some of the one or more benzimidazole compounds are in undissolved form suspended in the topical composition.

16. The method of claim 14 wherein the one or more benzimidazole compounds comprises mebendazole and wherein the inflammatory or autoimmune disease or condition comprises any form of rosacea.

17. The method of claim 16 wherein applying the topical composition is repeated at least once per day for a treatment period comprising at least two weeks; and
wherein applying the topical composition comprises applying around 0.025 to 0.5 g to the area of human skin; and
wherein the area of human skin comprises facial skin.

18. The method of claim 17 wherein the method achieves a reduction in a number of cutaneous cytotoxic CD+8 T-cells of 50% or greater in the area of human skin at an end of the treatment period compared to a number of cutaneous cytotoxic CD+8 T-cells in the area of human skin prior to the treatment period.

19. A method of making a mebendazole treatment composition, the method comprising:
adding an amount of one or more solvents and an amount of mebendazole to form a first mixture; and
heating the first mixture to a first temperature within a first temperature range for a first period of time while mixing or stirring to form a heated mixture;
wherein the amount of mebendazole is around 0.01-1.0% by weight of the mebendazole treatment composition;
wherein the first temperature range is at least 60-90° C.;
wherein the first period of time is 5 to 15 minutes;
wherein at least some of the mebendazole remains in undissolved form and suspended in the mebendazole treatment composition; and
wherein the mebendazole treatment composition does not include any aprotic solvents.

20. The method of claim 19 wherein the amount of mebendazole is 0.01% to 0.075%.

21. The method of claim 20 wherein the first temperature range is 70-90° C.

22. The method of claim 20 wherein the one or more solvents comprise a sorbitol based solvent, or a glycol based solvent, or both.

23. The method of claim 20 wherein the one or more solvents comprises dimethyl isosorbide.

24. The method of claim 20 wherein the one or more solvents comprises diethylene glycol monoethyl ether, or propylene glycol monolaurate, or both.

25. The method of claim 22 further comprising:
cooling the heated mixture to a second temperature within a second range of temperatures by adding water and mixing or stirring to form a cooled mixture;
wherein the second range of temperatures is around 35-50° C.; and
wherein the cooling the heated mixture is completed in 15 minutes or less.

26. The method of claim 25 further comprising dissolving an amount of a viscosity modifying agent or an amount of an anti-precipitation agent or both in the water prior to the cooling step;
wherein the viscosity modifying agent comprises hydroxyethylcellulose;
wherein the anti-precipitation agent comprises 2-10% hydroxypropyl beta-cyclodextrin by weight of the mebendazole treatment composition.

27. The method of claim 25 further comprising microfluidizing the heated mixture or the cooled mixture in a microfluidizer.

28. The method of claim 27 wherein the microfluidizing is carried out at a pressure of 5,000-20,000 psi and is repeated for at least three passes through the microfluidizer.
